(19) United States
(12) Patent Application Publication
(10) Pub. No.: US 2025/0281588 A1
(43) Pub. Date: Sep. 11, 2025

(54) PREVENTION AND TREATMENT OF INFECTIONS WITH INTRACELLULAR BACTERIA

(71) Applicants: UNIVERSITEIT GENT, Gent (BE); VIB VZW, Zwijnaarde (BE)

(72) Inventors: Rein Verbeke, Gent (BE); Ine Lentacker, Gent (BE); llke Aernout, Gent (BE); Stefaan De Smedt, Gent (BE)

(21) Appl. No.: 18/861,367

(22) PCT Filed: Apr. 27, 2023
(86) PCT No.: PCT/EP2023/061133

§ 371 Date: Oct. 29, 2024

(30) Foreign Application Priority Data
Apr. 29, 2022 (EP) 22170792.0

(51) Int. Cl. A61K 39/02; A61K 39/00; A61P 31/04

(52) U.S. Cl. CPC A61K 39/0208; A61P 31/04; A61K 2039/53; A61K 2039/54; A61K 2039/55555; A61K 2039/55572; A61K 2039/57

(57) ABSTRACT

The present invention relates to a method and composition for prevention and treatment of infections in a subject, in particular, with intracellular bacteria. The invention provides an optimized intracellular delivery of an active agent, in particular, a nucleic acid, using a nanoparticle formulation including an iNKT cell agonist.

### CROSS-REFERENCE TO RELATED APPLICATIONS

This application is a national phase entry under 35 U.S.C. § 371 of International Patent Application PCT/EP2023/061133, filed Apr. 27, 2023, designating the United States of America and published as International Patent Publication WO 2023/209103 A1 on Nov. 2, 2023, which claims the benefit under Article 8 of the Patent Cooperation Treaty of European Patent Application Serial No. 22170792.0, filed Apr. 29, 2022.

### STATEMENT REGARDING ELECTRONIC FILING OF A SEQUENCE LISTING

Pursuant to 37 C.F.R. § 1.831 through 1.835, a Sequence Listing XML file entitled “6XI5294-P2022027PCT-seql-000001” 7.29 kilobytes in size, generated Oct. 10, 2024, has been submitted via EFS-Web is provided in lieu of a paper copy, which Sequence Listing is hereby incorporated by reference into the specification.

### TECHNICAL FIELD

This disclosure relates to a method and composition for prevention and treatment of infections in a subject, in particular, with intracellular bacteria. The disclosure provides an optimized intracellular delivery of an active agent, in particular, a nucleic acid, using a nanoparticle formulation including an iNKT cell agonist.

### BACKGROUND

Antibiotic resistance is rising to dangerously high levels in all parts of the world. Several bacteria have evolved diverse immune escape strategies and overcome immune responses by residing and multiplying inside host immune cells, primarily macrophages. Failure of conventional antibiotic therapy is often encountered because infected cells prevent drug permeation or the drug concentration is too low at the site of resident bacteria. Additionally, with the emergence of multi-drug and extensively-drug resistant strains, new tools to control bacterial epidemics are urgently required.

Although vaccines are recognized as highly effective tools to mitigate antibiotic resistance, effective vaccine development for many bacteria is held back by a lack of conventional vaccine platforms struggling to elicit the required protective immune responses. The current SARS-COV-2 pandemic has demonstrated the safety and effectiveness of messenger RNA (mRNA) vaccines and confirmed their role as next generation vaccines (Verbeke et al., 2021). These vaccines contain mRNA encoding pathogen antigens complexed within lipid nanoparticles. While these mRNA vaccines can confer protection against SARS-COV-2 infection by providing potent neutralizing antibody responses, preventing or treating infections with intracellular bacteria might request a more multifaceted immune response, involving both humoral and cellular responses, as well as heterologous and long-term innate immune effects to achieve effective immunity. Despite a potential of mRNA vaccines for (intracellular) bacteria, only a handful of studies have investigated this promising avenue to date. Moreover, it is still debatable whether a mRNA vaccine platform, like the ones currently used in the licensed SARS-COV-2 vaccines, is sufficiently suitable to provide protective immunity against bacterial pathogens.

Verbeke et al. (2019) reports the design of mRNA and adjuvant-loaded lipid nanoparticles for therapeutic cancer vaccination, where vaccine-induced antitumor immune responses are empowered by an invariant Natural Killer T cell (INKT) agonist. The study, however, is silent on the treatment of infectious bacterial diseases.

In this disclosure, it is demonstrated that the inclusion of an iNKT agonist in an mRNA vaccine drastically increases the potency of mRNA vaccines to provide protective immunity against intracellular bacteria.

### BRIEF SUMMARY

This disclosure provides methods for preventing and/or treating an infection, more specifically by an intracellular bacterial pathogen in a subject, by administering a composition comprising adjuvant loaded nanoparticles to the subject. In some aspects and as shown in the below examples, it has been observed that use of the adjuvant-loaded nanoparticles of the disclosure showed a higher inhibition of infection of cells with an intracellular bacterium as compared to the use of nanoparticles without adjuvants.

In one embodiment, this disclosure relates to nanoparticles and uses thereof, wherein the nanoparticles are associated (e.g., complexed, conjugated, encapsulated, absorbed, adsorbed, admixed) with an iNKT cell agonist, in particular, a glycolipid, and a nucleic acid. The nanoparticles are used in methods of treating, preventing or ameliorating infections in a subject, in particular, of treating, preventing or ameliorating infections with intracellular bacteria.

In a further embodiment, the nanoparticle is a lipid-based nanoparticle and/or a cationic nanoparticle, in particular, a lipoplex particle or lipid nanoparticle, which encapsulates or comprises a nucleic acid encoding a bacterial antigen, more specific an antigen derived (i.e., originated) from an intracellular bacterium. In a further embodiment, the nanoparticle comprises a lipid component that includes at least one lipid, more specific a cationic lipid or an ionizable lipid, and at least one (helper) lipid, such as e.g., a phospholipid, a steroid or sterol, such as cholesterol or (functional) derivative or analogue thereof, and/or a PEG containing lipid. The nanoparticle is further associated with an adjuvant, in particular, an immunity stimulating adjuvant, more in particular, an iNKT cell agonist. More particular, the nanoparticle of the disclosure comprises or consists essentially of a lipid component, a nucleic acid encoding a bacterial antigen, more specific RNA, and an iNKT cell agonist, such as a glycolipid. In one embodiment, the glycolipid is an α-GalCer compound. Optionally, the RNA, in particular, mRNA, includes the part or complete incorporation of modified nucleosides such as pseudouridine (Ψ), N1-methylpseudouridine (m1Ψ), and/or 5-methyl uridine (5mU) into the mRNA transcript.

In another embodiment, the iNKT cell agonist, such as an α-GalCer compound, is incorporated in the lipid component of the nanoparticle. In particular, the concentration of the α-GalCer compound in the nanoparticle is between and about 0.0015 mol % and about 5 mol % of the total lipid amount (<1 μg/kg body weight).

The nucleic acid, such as mRNA, as provided herein encodes an antigen (e.g., a protein or peptide) of interest, in particular, an antigen derived from an infectious bacterium, and more specifically an intracellular bacterium. Optionally, the mRNA includes a chain terminating nucleoside, a polyA sequence, a polyadenylation signal, and/or a 5′ cap structure.

The disclosure also provides a pharmaceutical composition comprising the nanoparticle as provided herein and a pharmaceutically acceptable excipient, carrier and/or diluent.

In a further embodiment, the nanoparticle or the composition of this disclosure is used for preventing, ameliorating or treating an infection, in particular, an infection with an intracellular bacterial pathogen. More specific, the disclosure provides a method for delivering and/or expressing an antigen derived from an intracellular bacterium to antigen-presenting cells, preferably antigen-presenting cells in the spleen, lungs, liver and/or lymph nodes, the method comprising administering the nanoparticle or composition as provided herein. In a particular embodiment, the antigen-presenting cells are dendritic cells, B-cells, macrophages or monocytes.

Furthermore, the disclosure provides a nanoparticle or composition for use in a method for inducing an immune response, preferably an immune response against an infectious agent, in particular, an intracellular bacterial pathogen, in a subject, comprising administering to the subject the nanoparticle or composition as described herein. The nanoparticle or composition is able to stimulate, prime and/or expand T cell lymphocytes against a bacterial antigen, and/or elicit a humoral immune response (B cell response) resulting in the production of antibodies against a bacterial antigen, and/or enhance and broaden the induced innate immune response by the activation of iNKT cells in a subject.

In one embodiment, the nanoparticle or composition of the disclosure is administered in two, three or more (subsequent) doses to the subject, e.g., at least once a week, every two weeks, every month, etc. Administration can be intravenous, intramuscular, intradermal, subcutaneous, intraperitoneal, intratracheal, intranasal or via inhalation.

### BRIEF DESCRIPTION OF THE DRAWINGS

With specific reference to the figures, it is to be noted that the particulars shown are by way of example and for purposes of illustrative discussion of the different embodiments of this disclosure. The description taken with the figures make it apparent to those skilled in the art how the several forms of the disclosure may be embodied in practice.

FIG. 1: Incorporation of αGC in a mRNA-LNP based Listeria vaccine drastically increases the protection against listeria infection. C57BL/6 mice were intramuscularly vaccinated with a prime-boost regime (14 days interval) of 2 μg N1-methyl pseudo uridine (m1ψ) modified mRNA encoding a newly discovered listeria protein antigen (OppA lmon_0149) formulated in LNPs alone, or co-formulated with αGC adjuvant. The injection of PBS was included as negative control, while a low-dose infection with Listeria monocytogenes EGD (5×10⁴ CFUs) was included as positive control for successful immunization. Mice vaccinated with 2 μg OVA-mRNA (m1ψ) formulated in LNPs alone, or co-formulated with αGC adjuvant were included as additional controls. Two weeks after prime vaccination, an identical booster was administered and two weeks later the animals were challenged by intravenous injection of 7.5×10⁵ bacteria. Mice were euthanized 72 hours post-challenge and the bacterial load in spleen and liver was assessed by counting colony-forming units (CFU) after serial dilution and replating. Bar charts depicting the number of CFUs in liver (FIG. 1A) and spleen (FIG. 1B). (n=6, Mann-Whitney test, ** p value<0.01).

FIG. 2: C57BL/6 mice were intravenously vaccinated with a prime-boost regime (14 days interval) of 10 μg N1-methyl pseudo uridine (m1ψ) modified mRNA encoding OppA 1mon_0149 formulated in lipoplexes and co-formulated with αGC adjuvant. The injection of PBS was included as negative control, while a low-dose infection with Listeria monocytogenes EGD (1×10⁴ CFUs) was included as positive control for successful immunization. Two weeks after prime vaccination, an identical booster was administered and two weeks later the animals were challenged by intravenous injection of 7.5×10⁵ bacteria. Mice were euthanized 72 hours post-challenge and the bacterial load in spleen and liver was assessed by counting colony-forming units (CFU) after serial dilution and replating. Bar charts depicting the number of CFUs in liver (FIG. 2A) and spleen (FIG. 2B). (n=3-5, Mann-Whitney test compared to negative PBS control, ** p value<0.01).

FIG. 3: Cytotoxic T cell responses in spleen upon prime-boost vaccination against the model antigen OVA. C57BL/6 mice were immunized intravenously (IV), intramuscularly (IM) or subcutaneously (SC) with a prime-boost dose (14 days interval) of 10 μg OVA-mRNA (m1ψ) formulated in LNPs alone, or co-formulated with αGC adjuvant (n=4-5). (FIG. 3A) OVA-specific CD8+ T cells in spleen were identified using an H-2Kb OVA tetramer, two weeks after the boost vaccine dose. (FIG. 3B) IFN- [symbol] producing CD8+ T cells were detected after restimulating splenocytes with the OVA-derived peptide SIINFEKL (SEQ ID NO:1). Statistical analysis was performed by a one-way ANOVA followed by Tukey's post hoc test. Asterisks indicate statistical significance compared to the untreated group (*, p<0.05; **, p<0.01; ***, p<0.001).

FIG. 4: Th1 CD4 T cell responses against OVA upon prime-boost vaccination. C57BL/6 mice were immunized intravenously (IV), intramuscularly (IM) or subcutaneously (SC) with a prime-boost dose (14 days interval) of 10 μg OVA-mRNA (ml) formulated in LNPs alone, or co-formulated with αGC adjuvant (n=5). Figure depicts the frequency of IFN-y producing CD4+ T cells after restimulating splenocytes with the OVA-derived peptide ISQAVHAAHAEINEAGR (SEQ ID NO:2). Statistical analysis was performed by a one-way ANOVA followed by Tukey's post hoc test. Asterisks indicate statistical significance compared to the untreated group (*, p<0.05; **, p<0.01; ***, p<0.001, **** p<0.0001).

FIG. 5: Antibody titers against OVA upon prime-boost vaccination. C57BL/6 mice were immunized intravenously (IV), intramuscularly (IM) or subcutaneously (SC) with a prime-boost dose (14 days interval) of 10 μg OVA-mRNA (mlv) formulated in LNPs alone, or co-formulated with αGC adjuvant (n=5). Sera were collected two weeks after the second vaccine dose. (FIG. 5A) Anti-OVA IgG1 and (FIG. 5B) anti-OVA IgG2c responses determined by ELISA. Statistical analysis was performed by a one-way ANOVA followed by Tukey's post hoc test. Asterisks indicate statistical significance compared to the untreated group (*, p<0.05; **, p<0.01; ***, p<0.001).

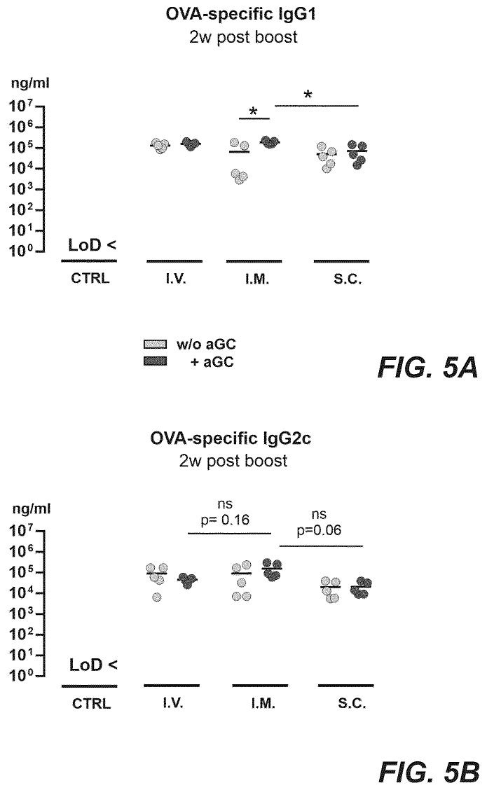

FIG. 6: Dose reduction study indicating dose sparing effects of αGC adjuvant for mRNA-LNP vaccines to induce cytotoxic T cell responses. C57BL/6 mice were immunized intramuscularly with a single mRNA dose of 5 μg, 2.5 μg or 0.5 μg OVA-mRNA (mlv) formulated in LNPs alone, or co-formulated with αGC adjuvant (n=3). OVA-specific CD8+ T cells in spleen were identified using an H-2Kb OVA tetramer, two weeks after vaccination.

FIG. 7: C57BL/6 mice were IM immunized with a prime-boost dose (14 days interval) of Sig-tOVA-LAMP1 mRNA or SP-tOVA-MITD mRNA (2 μg mRNA dose) alone, or in combination with αGC. OVA-specific CD8+ T cells in spleen were identified using an H-2Kb OVA tetramer, two weeks after vaccination (n=6, Mann-Whitney test, * p value<0.05).

FIG. 8: Vaccine induced CD4+ T helper response against the Mycobacterium tuberculosis antigen; ESAT-6. C57BL/6 mice were IM immunized with a prime-boost dose (14 days interval) of SP-ESAT-6-MITD mRNA (2 μg mRNA dose) alone, or in combination with αGC. (FIG. 8A) ESAT-6 specific CD4+ T cells in spleen identified using I-A (b)/4-17 QQWNFAGIEAAASA tetramer. (FIG. 8B) Cytokine secretion upon 24 hours restimulation of splenocytes with ESAT-6 protein, including the detection of IFN-Y, IL-2, IL-4, IL-6 and IL-22. Statistical analysis was performed by Kruskal-Wallis test, followed by Dunn's posthoc test (*, p<0.05; **, p<0.01; ***, p<0.001).

FIG. 9: (FIG. 9A) NKT cell response measured in draining lymph node. C57BL/6 mice were immunized intramuscularly with a single mRNA dose of a mixture of 8 μg OVA and firefly luciferase mRNA (m1ψ) formulated in LNPs alone, or co-formulated with αGC adjuvant (˜25ng aGC/mouse). Three days and 7 days post-injection, NKT cell frequencies were measured in the draining inguinal lymph node. (FIG. 9B) Representative flow plots of NKT cells (TCRβ+, mCD1d PBS-57+ cells). Additional surface markers were used to detect a follicular helper-like NKT cell phenotype (PD-1, CXCR5). Percentages (FIG. 9C) and representative flow plots (FIG. 9D) of follicular-like NKT cells among total NKT cells. (n=3−4). Statistical analysis was performed by a one-way ANOVA followed by Tukey's post hoc test. Asterisks indicate statistical significance compared to the untreated group (*, p<0.05; **, p<0.01; ***, p<0.001).

FIG. 10: Cytokine responses measured in blood serum. C57BL/6 mice were immunized intramuscularly with a single mRNA dose of 8 μg OVA-mRNA (mlv) formulated in LNPs alone, or co-formulated with αGC adjuvant (˜25ng αGC/mouse). Four hours or 24 hours post-injection, sera was collected and screened for the release of inflammatory cytokines. Statistical analysis was performed by a one-way ANOVA followed by Tukey's post hoc test. Asterisks indicate statistical significance compared to the untreated group (*, p<0.05; **, p<0.01; ***, p<0.001).

### DETAILED DESCRIPTION

This disclosure will now be further described. In the following passages, different aspects of the disclosure are defined in more detail. Each aspect so defined may be combined with any other aspect or aspects unless clearly indicated to the contrary. As used in the specification and the claims, the singular forms “a,” “an,” and “the” include plural referents unless the context clearly dictates otherwise. By way of example, “a compound” means one compound or more than one compound. Throughout the description and claims of this specification the word “comprise” and other forms of the word, such as “comprising” and “comprises,” means including but not limited to, and is not intended to exclude, for example, other additives, components, integers, or steps. As used herein, “about,” “approximately,” “substantially,” and “significantly” will be understood by persons of ordinary skill in the art and will vary to some extent on the context in which they are used. If there are uses of the term that are not clear to persons of ordinary skill in the art given the context in which it is used, “about” and “approximately” will mean plus or minus 10% of the particular value or term. The term “consisting essentially of” or “consists essentially of” means that e.g., a product or method must contain the listed compounds, ingredient(s), or steps and may also contain small amounts (for example, up to 5% by weight, or up to 1% or 0.1% by weight) of other ingredient(s), compounds, or steps provided that any additional ingredients, compounds, or steps do not affect the essential properties of the respective product or method. The term allows for elements not explicitly recited, but exclude elements that are found in the prior art or that affect a basic or novel characteristic of the disclosure. The terms described above and others used in the specification are well understood to those in the art. All references, and teachings specifically referred to, cited in the present specification are hereby incorporated by reference in their entirety.

This disclosure is generally directed to nanoparticles, compositions and vaccines for preventing, ameliorating or treating infections or infectious diseases caused by intracellular bacteria in humans and animals. The term “infectious disease” as used herein refers to any kind of clinically evident disease resulting from the presence of a pathogen or germ, in particular, pathogenic bacteria, more in particular, intracellular pathogenic bacteria. Specifically, this disclosure is directed at optimizing pathogen antigen presentation and innate immune stimulation to enable the recipient to generate the maximum immune response to pathogen-derived and pathogen-specific proteins or antigens.

The disclosure provides specifically designed nanoparticles and their use in the prevention, reduction or treatment of infections with intracellular bacterial pathogens. In particular, it was demonstrated for the first time that the nanoparticles as provided herein, including compositions comprising them, target antigen presenting cells and evoke an improved antigen-specific immune response against the pathogen. More particular, it was shown that the combination of a nanoparticle with an iNKT cell agonist and an mRNA encoding an antigen from an intracellular bacterial pathogen, provided synergistic protective effects against infection with an intracellular bacterium. Remarkably, nanoparticles comprising mRNA encoding a bacterial antigen and an iNKT cell agonist, are about 3- to 50-fold more potent to provide protective immunity against an intracellular bacterial infection, than nanoparticles without the iNKT cell agonist. More specific, the combination or complexation of a nanoparticle with mRNA and the iNKT ligand such as e.g., an α-GalCer compound (incorporated at very low concentrations) showed increased potency to induce cellular immunity, compared to the use of nanoparticles without adjuvants, evidenced by 1.5-to-5 fold higher cytotoxic CD8 T cell responses and 2-to-3 fold higher Th1-polarized (IFN- [symbol] producing) CD4 T cell responses. Moreover, inclusion of the iNKT cell agonist in a mRNA vaccine increased vaccine-induced IgG1 antibody titers against the mRNA-encoded antigen 2-to-3 fold.

In addition and of particular interest is that the presence of an iNKT ligand in the nanoparticle such as e.g., an α-GalCer compound (incorporated at very low concentrations) showed a dose-reduction potential for a mRNA vaccine to elicit cytotoxic T cells. In a particular embodiment, nanoparticles comprising the iNKT cell agonist required a much lower effective mRNA dose to induce cytotoxic CD8 T cells than nanoparticles without the agonist, i.e., a 2-to-10 fold reduction, in particular, a 2-to 5-fold reduction. This is a clear advantage in dose-sparing strategies.

Furthermore, inclusion of an iNKT cell agonist (e.g., α-GalCer) in a mRNA vaccine resulted in a more diverse CD4 T cell response against an mRNA-encoded intracellular bacteria antigen, including induction of T helper 1 (Th1), Th2, Th17 and Th22 cytokine responses. Certain types of Th responses have been shown to play a crucial role in protective immunity against intracellular bacteria, such as e.g., Mycobacterium tuberculosis and Chlamydia trachomatis.

In an aspect of this disclosure, it was shown that the combination of a nucleic acid encoding an intracellular bacterial antigen with the iNKT ligand, such as e.g., an α-GalCer compound (incorporated at very low concentrations), not only promotes adaptive immunity, but also offers the advantage of activating NKT cells resulting in an enhanced and broader innate immune activation, evidenced by proliferation of NKT cells in targeted immune organs and rapid, but temporal production of a broad scale of cytokines that can attribute to protective immunity against intracellular bacteria, for example, IFN- [symbol] , IL-22, and IL-17 cytokines.

In general, antigen presentation encompasses a group of variables that determine how a recipient processes and responds to an antigen. These variables can include, but are not limited to, adjuvants, vaccine component concentration, carrier molecules, haptens, dose frequency and route of administration. This disclosure provides an effective approach to present bacterial (extracellular and/or immunogenic) proteins and/or peptides to a subject and to induce remarkably robust protective immune responses thereto.

As used herein, ‘intracellular’ bacteria or bacterial pathogens refers to (micro) organisms (also referred to as infectious agents), that have the capacity to invade and survive within eukaryotic host cells. It includes facultative and obligate intracellular bacteria. In a particular embodiment the bacterium is a facultative intracellular bacterium. Several intracellular bacterial pathogens thrive inside mononuclear phagocytes such as macrophages and dendritic cells (DCs). Alternatively, the endosomal compartment or the cytosol of host cells such as neutrophils, fibroblasts, or epithelial cells serves as important habitat for certain intracellular bacterial pathogens. Intracellular persistent infections change the nature of the host, alter immune function and immunological protection, and predispose the host to other persistent infections.

As such, the nanoparticles of this disclosure are ideally suited as effective vaccines or therapies for use against a variety of intracellular bacteria such as Listeria sp., Mycobacterium sp., Legionella sp., Salmonella sp., Chlamydia sp., Rickettsia sp., Brucella sp., Staphylococcus sp., Streptococcus sp., Enterococcus sp., Shigella sp., Coxiella sp., Helicobacter sp., Campylobacter sp., Ehrlichia sp., Yersinia sp., Borrelia sp., Rhodococcus sp., Bartonella sp., Acinetobacter sp., Escherichia sp., Clostridium sp. and Veillonella sp. In a particular embodiment, the nanoparticles of the disclosure are used to treat infections with obligate or facultative anaerobic Gram-positive bacteria, e.g., selected from the group consisting of Listeria sp., Mycobacterium sp., Enterococcus sp., Streptococcus sp., Staphylococcus sp., Clostridium sp. and Rhodococcus sp. In another embodiment, the nanoparticles of the disclosure are used to treat infections with obligate or facultative anaerobic Gram-negative bacteria, e.g., selected from the group consisting of Chlamydia sp., Legionella sp., Salmonella sp., Escherichia sp., Yersinia sp., Rickettsia sp., Brucella sp., Shigella sp., Coxiella sp., Helicobacter sp., Campylobacter sp., Ehrlichia sp., Bartonella sp., Acinetobacter sp., Veillonella sp., and Borrelia sp.

Exemplary species to be treated include Listeria monocytogenes, Mycobacterium bovis, Mycobacterium tuberculosis, Mycobacterium leprae, Mycobacterium Ulcerans, Legionella pneumophila, Salmonella typhimurium, Salmonella enteritidis, Chlamydia pneumonia, Chlamydia trachomatis, Chlamydia psittaci, Neisseria meningitides, Rickettsia rickettsia, Rickettsia typhi, Brucella melitensis, Staphylococcus aureus, Streptococcus pyogenes, Enterococcus faecalis, Shigella dysenteriae, Coxiella burnetii, Helicobacter pylori, Campylobacter jejuni, Ehrlichia chaffeensis, Ehrlichia phagocytophila, Burkholderia pseudomallei, Yersinia pestis, Borrellia Burgdorferi, Borrellia Mayonii, Rhodococcus equi, Bartonella henselae, Acinetobacter baumannii, Escherichia coli and Clostridium botulinum, Clostridium difficile, Clostridium perfringens and Clostridium tetani. In a specific embodiment, the species to be treated is Listeria monocytogenes. In another embodiment, the species to be treated is Mycobacterium tuberculosis.

Each nanoparticle, composition or vaccine of this disclosure can comprise or express at least one agent, in particular, an epitope or antigen specific for a given bacterial species or strain, including combinations thereof. This agent may be a nucleic acid or a protein or polypeptide, preferably a nucleic acid encoding a protein or polypeptide, in particular, an antigen derived from an intracellular bacterium. The antigen may be a protein or antigenic fragment thereof from bacterial pathogens as provided herein. Alternatively, the antigen may be a synthetic gene, constructed using recombinant DNA methods, which encode antigens or parts thereof from bacterial pathogens as provided herein. These pathogens can be infectious in humans, domestic animals or wild animal hosts. The antigen can be any molecule, in particular, a protein or peptide, that is expressed by the bacterial pathogen prior to or during entry into, colonization of, or replication in the host. The terms “protein” or “polypeptide” generally encompass proteins encoded by any open reading frame (ORF) of a genome. Where a single ORF encodes a pre-protein that is processed into one, two or more mature proteins, the term may encompass both the pre-protein and the processed mature proteins. The term “nucleic acid” as used herein means a polymer of any length composed essentially of nucleotides, e.g., deoxyribonucleotides and/or ribonucleotides. The term “nucleic acid” further encompasses DNA, RNA and DNA/RNA hybrid molecules, specifically including circRNA, hnRNA, tRNA, rRNA, ncRNA, IncRNA, miRNA, siRNA, pre-mRNA, mRNA, cDNA, genomic DNA, amplification products, oligonucleotides, and synthetic (e.g., chemically synthesized) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesized. A “nucleic acid” can be double-stranded, partly double stranded, or single-stranded. Where single-stranded, the nucleic acid can be the sense strand or the antisense strand. In addition, nucleic acid can be circular or linear. By “encoding” is meant that a nucleic acid sequence or part(s) thereof corresponds, by virtue of the genetic code of an organism in question, to a particular amino acid sequence, e.g., the amino acid sequence of one or more desired proteins or (poly)peptides.

In a preferred embodiment, the nucleic acid is DNA or RNA, particularly RNA, more particular messenger RNA (mRNA). More specifically, the nucleic acid is mRNA comprising a coding sequence (CDS) encoding the desired protein or peptide, in particular, the bacterial antigen. Nucleic acid sequences derived from a particular (wild type) nucleic acid sequence may be variants of that particular sequence or a fragment thereof. For example, it will be understood by one of ordinary skill in the art that the peptides or proteins suitable for vaccination herein may be altered such that they vary in sequence from the naturally occurring or native target sequences from which they were derived, while retaining the desirable activity to induce an immune response against the native sequences. In another embodiment, the nucleic acid sequence may be codon-optimized. A protein encoded by an mRNA may be of any size and may have any secondary structure or activity. In certain embodiments, the nucleic acid has an open reading frame (ORF) encoding an antigen of interest, in particular, an antigen from an intracellular bacterium.

The term “bacterial antigen” refers to any bacterial component having antigenic properties, i.e., being able to provoke an immune response in an individual and will be selected by the skilled person. The bacterial antigen may be an intracellular antigen, cell-wall or surface antigen or secreted antigen from the bacterium. Typically, the antigen has no known toxicity or enzymatic activity. In a specific embodiment, the antigen is derived from a bacterium, in other words a bacterial antigen, in particular, from an intracellular bacterium, more in particular, a bacterial species selected from the group consisting of: Listeria sp., Mycobacterium sp., Legionella sp., Salmonella sp., Chlamydia sp., Rickettsia sp., Brucella sp., Staphylococcus sp., Streptococcus sp., Enterococcus sp., Shigella sp., Coxiella sp., Helicobacter sp., Campylobacter sp., Ehrlichia sp., Yersinia sp., Borrelia sp., Rhodococcus sp., Bartonella sp., Acinetobacter sp., Escherichia sp., Clostridium sp. and Veillonella sp. More specific, the antigen is from a bacterial species selected from the group consisting of Listeria monocytogenes, Mycobacterium bovis, Mycobacterium tuberculosis, Mycobacterium leprae, Mycobacterium Ulcerans, Legionella pneumophila, Salmonella typhimurium, Salmonella enteritidis, Chlamydia pneumonia, Chlamydia trachomatis, Chlamydia psittaci, Neisseria meningitides, Rickettsia rickettsia, Rickettsia typhi, Brucella melitensis, Staphylococcus aureus, Streptococcus pyogenes, Enterococcus faecalis, Shigella dysenteriae, Coxiella burnetii, Helicobacter pylori, Campylobacter jejuni, Ehrlichia chaffeensis, Ehrlichia phagocytophila, Burkholderia pseudomallei, Yersinia pestis, Borrellia Burgdorferi, Borellia Mayonii, Rhodococcus equi, Bartonella henselae, Acinetobacter baumannii, Escherichia coli and Clostridium botulinum, Clostridium difficile, Clostridium perfringens and Clostridium tetani.

In one embodiment, the bacterial antigen for use in the methods of the disclosure and for preventing, reducing or treating an infection with Mycobacterium tuberculosis is selected from the group consisting of: MTB32A (Rv0125/UniProtKB Acc. no. 007175), AG58C/FbpC (Rv0129c/UniProtKB Acc. no. P9WQN9), McelA (Rv0169/UniProtKB Acc. nos. F5BH89 and Q79FZ9), EsxG/TB9.8 (Rv0287/UniProtKB Acc. no. 053692), CFP-7/EsxH/TB10.4 (Rv0288/UniProtKB Acc. no. P9WNK3), GroEL2/hsp65 (Rv0440/UniProtKB Acc. no. P9WPE7), protease heat shock protein X (Rv0563/UniProtKB Acc. no. P9WHS5), (Rv0569/UniProtKB Acc. no. P9WM83), RplJ (Rv0651/UniProtKB Acc. no. P9WHE7), PPE12 (Rv0755c/UniProtKB Acc. no. P9WI37), KdpC (Rv1031/UniProtKB Acc. no. P9WKF1), PPE15 (Rv1039c/UniProtKB Acc. no. P9WI31), (Rv1085c/UniProtKB Acc. no. P9WFN7), MTB39A/PPE18 (Rv1196/UniProtKB Acc. no. L7N675), EsxL (Rv1198/UniProtKB Acc. no. P9WNJ5), CysD (Rv1285/UniProtKB Acc. no. P9WIK1), MPT32/ModD/Apa (Rv1860/UniProtKB Acc. no. P9WIR7), Ag85B/FbpB (Rv1886c/UniProtKB Acc. no. P9WQP1), Mpt63 (Rv1926c/UniProtKB Acc. no. P9WIP1), Mpt64 (Rv1980c/UniProtKB Acc. no. P9WIN9), (Rv2016/UniProtKB Acc. no. O53462), PfkB (Rv2029c/UniProtKB Acc. no. P9WID3), HspX (Rv2031c/UniProtKB Acc. no. P9WMK1), PPE39 (Rv2353c/UniProtKB Acc. no. Q79FF3), rpfD (Rv2389c/UniProtKB Acc. no. P9WG27), fAS (Rv2524c/UniProtKB Acc. no. P95029), PPE42 (Rv2608/UniProtKB Acc. no. P9WHZ5), Hrpl (Rv2626c/UniProtKB Acc. no. P9WJA3), (Rv2660c/UniProtKB Acc. no. 16Y1F5), MPT83/lipoprotein P23 (Rv2873/UniProtKB Acc. no. P9WNF3), MPT70 (Rv2875/UniProtKB Acc. no. P9WNF5), EsxR/TB10.3 (Rv3019c/UniProtKB Acc. no. P9WNI9), PPE51(Rv3136/UniProtKB Acc. no. P9WHY3), PPE52 (Rv3144c/UniProtKB Acc. no. 16X6H8), GroES/mpt 57/cpn10 (Rv3418c/UniProtKB Acc. no. P9WPE5), PPE60/mtb39c (Rv3478/UniProtKB Acc. no. Q6MWX1), OtsA (Rv3490/UniProtKB Acc. no. P9WN11), Mce4C (Rv3497c/UniProtKB Acc. no. 16YGB1), EspC (Rv3615c/UniProtKB Acc. no. P9WJD7), EspA (Rv3616c/UniProtKB Acc. no. P9WJE1), EsxV (Rv3619c/UniProtKB Acc. no. PODOA7), EsxW (Rv3620c/UniProtKB Acc. no. P9WNI3), Mpt51/fbpC1 (Rv3803c/UniProtKB Acc. no. P9WQN7), Secreted 85-a/FbpA (Rv3804c/UniProtKB Acc. no. P9WQP3), PE35 (Rv3872/UniProtKB Acc. no. P9WIG7), PPE68 (Rv3873/UniProtKB Acc. no. P9WHW9), CFP-10/EsxB (Rv3874/UniProtKB Acc. no. P9WNK5), ESAT-6/EsxA (Rv3875/UniProtKB Acc. no. P9WNK7), and EspI (Rv3876/UniProtKB Acc. no. P9WJC5).

In one embodiment, the disclosure provides a complex of a nanoparticle as provided herein and mRNA, and more specific an mRNA loaded nanoparticle. In some embodiments, the mRNA encodes one or multiple proteins or peptides. The mRNA may be designed to encode proteins or peptides of interest selected from any of several target categories including, but not limited to any naturally or non-naturally occurring or otherwise modified protein or peptide epitope(s). Two forms of mRNA structures can be used, i.e., conventional or non-replicating mRNA and self-amplifying mRNA. As known to the skilled person, in the conventional mRNA form, the antigen of choice is flanked by UTR regions, a 3′ poly(A) tail and a 5′ cap. Self-amplifying mRNA (saRNA) is based on the addition of a viral replicase gene to enable the mRNA to self-replicate. In addition, trans-amplifying mRNA (taRNA) is a new structural modality of mRNA vaccines and can be used in a further embodiment. The taRNA results from the splitting of the self-amplifying mRNA in a system with two templates, one containing the gene of interest and a second containing the replicase system. An mRNA may be a naturally or non-naturally occurring mRNA. An mRNA may include one or more modified nucleobases, nucleosides, or nucleotides. A nucleobase of a mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction.

The mRNA may include a 5′ untranslated region, a 3′ untranslated region, and/or a coding or translating sequence. Optionally, the mRNA includes one or more of a stem loop, a chain terminating nucleoside, a poly A sequence, a polyadenylation signal, and/or a 5′ cap structure.

A mRNA may include any number of base pairs, including tens, hundreds, or thousands of base pairs. Any number (e.g., all, some, or none) of nucleobases, nucleosides, or nucleotides may be an analogue of a canonical species, substituted, modified, or otherwise non-naturally occurring.

Substitutions and modifications to the mRNA of this disclosure may be performed by methods readily known to one of ordinary skill in the art.

In one embodiment, the nucleic acid can comprise at least one modification that confers increased or enhanced stability to the nucleic acid, including, for example, improved resistance to nuclease digestion in vivo. As used herein, the terms “modification” and “modified” as such terms relate to the nucleic acids provided herein, include at least one alteration that preferably enhances stability and renders the nucleic acid, such as an mRNA, more stable (e.g., resistant to nuclease digestion) than the wild-type or naturally occurring version of the nucleic acid. As used herein, the terms “stable” and “stability” as such terms relate to the nucleic acids of this disclosure, and particularly with respect to the mRNA, refer to increased or enhanced resistance to degradation by, for example, nucleases (i.e., endonucleases or exonucleases) that are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. Also contemplated by the terms “modification” and “modified” as such terms related to the mRNA of this disclosure are alterations that improve or enhance translation of mRNA nucleic acids readily known to one of ordinary skill in the art, including, for example, the inclusion of sequences that function in the initiation of protein translation (e.g., the Kozac consensus sequence).

In another embodiment, the RNA, in particular, mRNA, described herein may have modified nucleosides. In some embodiments, the mRNA comprises a modified nucleoside in place of at least one uridine. In some embodiments, mRNA comprises a modified nucleoside in place of each uridine. In particular, the modified nucleoside is independently selected from pseudouridine (ψ), N1-methyl-pseudouridine (m1ψ), N1-ethyl-pseudouridine, and 5-methyl-uridine (m5U), or combinations thereof. In some embodiments, the modified nucleoside comprises or consists of pseudouridine (ψ). In some embodiments, the modified nucleoside comprises or consists of N1-methyl-pseudouridine (m1ψ). In some embodiments, In some embodiments, the modified nucleoside comprises or consists of N1-ethyl-pseudouridine. In some embodiments, the modified nucleoside comprises or consists of 5-methyl-uridine (m5U). In some embodiments, the RNA may comprise more than one type of modified nucleoside mentioned herein. In some embodiments, the modified nucleoside replacing one or more uridine in the RNA may be any one or more of 3-methykiridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethyl-2-thio-uridine(Tm5s2U), 1-taurinomethyl-4-thio-pseudouridine, 5-methyl-2-thio-uridine (m5s2U), 1-methyl-4-thio-pseudouridine (m1s4ip), 4-thio-1-methyl-pseudouridine, 3-methyl-pseudouridine (m3ψ), 2-thio-1-methylpseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3 ψ), 5-(isopentenylaminomethyl) uridine (inm5U), 5-(isopentenylaminomethyl)-2-thio-uridine (inm5s2U), a-thio-uridine, 2′-O-methyl-uridine (Um), 5,2′-O-dimethyl-uridine (m5Um), 2′-O-methyl-pseudouridine (m), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2-F-uridine, 2′-OH-ara-uridine, 5-(2-carbomethoxyvinyl) uridine, 5-3-(1-E-propenylamino) uridine, or any other modified uridine known in the art.

By using the nucleoside-modified mRNA, the intracellular mRNA recognition by TLR3, TLR7, and TLR8 can be reduced, which makes the mRNA ‘immunosilent’ and avoids the release of type I IFNs. Despite an increase in translation capacity by uridine modifications, this comes together with a loss of RNA's self-adjuvant-effect, affecting hence DC activation and T-cell priming. In this disclosure it is demonstrated that the incorporation of a low amount of an iNKT cell agonist, more in particular, α-GalCer or analogue, ensures both a high antigen expression as well as a strong immune activation but without the strong induction of type I IFNs.

In a particular embodiment, the nanoparticle of this disclosure comprises or consists essentially of a lipid component, an iNKT cell agonist and an mRNA encoding an antigen derived from an intracellular bacterium, wherein the mRNA comprises a nucleoside-modified mRNA, more specific mRNA wherein one or more uridines are replaced by pseudouridine (ψ) N1-methyl-pseudouridine (m1ψ), N1-ethyl-pseudouridine, or 5-methyl-uridine (m5U), in particular, by N1-methyl-pseudouridine(s). In particular, at least 10% and up to all of the original uridines in the mRNA are substituted (e.g., 10-100%, 20-100%, 30-100%, 40-100%, 50-100%, 60-100%, 70-100%, etc.).

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C′s) and/or uridines (U′s) residues has been demonstrated, and RNA devoid of C and U residues have been found to be stable to most RNases. In some embodiments, the number of C and/or U residues in an mRNA sequence is reduced. In a another embodiment, the number of C and/or U residues is reduced by substitution of one codon encoding a particular amino acid for another codon encoding the same or a related amino acid. The term modification also includes, for example, the incorporation of non-nucleotide linkages or modified nucleotides into the mRNA sequences of this disclosure (e.g., modifications to one or both the 3′ and 5′ ends of an mRNA molecule encoding a functional secreted protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements that change the structure of an mRNA molecule (e.g., that form secondary structures).

This disclosure provides a nanoparticle formulation that successfully co-delivers bacterial antigen-encoding nucleic acids provided herein and an adjuvant to induce a pluripotent innate and adaptive antigen specific immune response in a subject, and more particular showing specific CD8+ T-cell responses and high levels of protection in vaccination challenge experiments in mice infected with intracellular bacteria. In its general composition, the formulation comprises at least the following elements:
a nanoparticle;
an antigen derived from an intracellular pathogen (e.g., a bacterial antigen), and more specific a nucleic acid encoding such antigen; and
an an iNKT cell agonist.

The term “nanoparticle” as used herein can be interpreted broadly and refers to a carrier being used as a transport module for another substance, such as a drug, in particular, a nucleic acid, more in particular, RNA, even more particular mRNA. Nanoparticles are currently being studied for their use in e.g., drug delivery and range from sizes of diameter 5-1000 nm, in particular, from about 5 to about 500 nm, more in particular, from about 5 to about 400 nm. In certain embodiments, the nanoparticle as envisaged herein has a mean diameter of 10-800 nm, 20-600 nm, 30-400 nm, or 40-300 nm, such as a mean diameter of 50-300 nm, 60-300 nm, 70-300 nm, 80-300 nm, 90-300 nm or 100-300 nm. The term “average diameter” refers to the mean hydrodynamic diameter of particles as measured by dynamic laser light scattering (DLS) with data analysis using the so-called cumulant algorithm, which provides as results the so-called Zaverage with the dimension of a length, and the polydispersity index (PI), which is dimensionless (Koppel, D., J. Chem. Phys. 57, 1972, pp 4,814-4820, ISO 13321). Here, “average diameter,” “diameter” or “size” for particles is used synonymously with this value of the Zaverage.

In particular, the size of the nanoparticle is such that it is capable of being taken up by a mammalian cell, in particular, an antigen presenting cell such as e.g., a dendritic cell. In one embodiment, the nanoparticle is a cationic nanoparticle. The term “cationic nanoparticle” refers to a nanoparticle comprising a cationic agent embedded in the core or at the surface. Where the nanoparticle is to be used for complexation of nucleic acids as a therapeutic agent, the positively charged nanoparticle is believed to interact electrostatically with the negatively charged DNA/RNA molecules, which not only facilitates complexation of the agent, but which may also protect the latter from enzymatic degradation.

The nanoparticle can be used to deliver nucleic acids to a target site of interest (e.g., cell, tissue, organ, and the like). The particle may be formed from at least one cationic or cationic ionizable lipid or lipid-like material, and/or at least one cationic polymer, or a mixture thereof, and a nucleic acid, more specific RNA. Cationic or cationic ionizable lipid or lipid-like material and/or the cationic polymer combine together with the nucleic acid to form aggregates, and this aggregation results in colloidal stable particles. In a further embodiment, particles described herein further comprise at least one lipid or lipid-like material other than a cationic or cationic ionizable lipid or lipid-like material, and/or at least one polymer other than a cationic polymer, or a mixture thereof.

Cationic agents/materials contemplated for use herein include those that are able to electrostatically bind nucleic acids. In some embodiments, cationic polymeric materials contemplated for use herein include any cationic polymeric materials with which nucleic acid can be associated, e.g., by forming complexes with the nucleic acid or forming vesicles in which the nucleic acid is enclosed or encapsulated.

In one embodiment, the cationic agent may be a polycationic agent such as but not limited to chitosan, peptides (such as poly(L-lysine)), peptide derivatives (such as poly(L-lysine)-palmitic acid), polyethylenimine (PEI), poly(amido ethylenimine), and poly(amido amine). Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. In addition, polymeric materials have been synthesized specifically for nucleic acid delivery. In one embodiment, a polymeric material may be or comprise protamine or polyalkyleneimine, in particular, protamine.

Poly(P-amino esters), in particular, have gained widespread use in nucleic acid delivery owing to their ease of synthesis and biodegradability. In some embodiments, such synthetic materials may be suitable for use as cationic materials herein. A “polymeric material,” as used herein, is given its ordinary meaning, i.e., a molecular structure comprising one or more repeat units (monomers), connected by covalent bonds. In some embodiments, such repeat units can all be identical; alternatively, in some cases, there can be more than one type of repeat unit present within the polymeric material. In some cases, a polymeric material is biologically derived, e.g., a biopolymer such as a protein.

A particular polycationic agent is a polymer, preferably a polysaccharide, more preferably dextran, which is functionalized with a reactive (meth)acrylate moiety and subsequently co-polymerized with a cationic (meth)acrylate monomer such as 2-aminoethyl methacrylate, 2-(diethylamino)ethyl methacrylate, 2-(dimethylamino)ethyl methacrylate, 2-N-morpholinoethyl methacrylate, 2-(tert-butylamino)ethyl methacrylate, 2-(diisopropylamino)ethyl methacrylate, or [2-(methacryloyloxy)-ethyl] trimethylammonium chloride.

In a further embodiment, the nanoparticle of the disclosure is a carrier or particle comprising a lipid component (so called lipid-based nanoparticles), also referred to as a lipoplex formulation or lipid-based nanoparticle, and includes lipid nanoparticles (LNP), liposomes and micelles. The use of lipid-based nanoparticles to facilitate the delivery of the nucleic acids provided herein, and especially RNA, more particular mRNA, to target cells is especially contemplated by this disclosure. The incorporated nucleic acids may be completely or partially located in the interior space of the particle, within the bilayer membrane of the particle, or associated with the exterior surface of the particle membrane. The association of a nucleic acid with a nanoparticle is also referred to herein as “encapsulation” wherein the nucleic acid is entirely integrated into the particle. The particle protects the nucleic acid from an environment that may contain enzymes or chemicals and allow the encapsulated nucleic acid to reach the target cell. While the nanoparticle can facilitate introduction of nucleic acids into target cells, the addition of polycations as provided herein, as a copolymer can facilitate, and in some instances markedly enhance the transfection efficiency.

The terms “lipid” and “lipid-like material” are used herein to refer to molecules that comprise one or more hydrophobic moieties or groups and optionally also one or more hydrophilic moieties or groups. Molecules comprising hydrophobic moieties and hydrophilic moieties are also frequently denoted as amphiphilic. Specific examples of amphiphilic compounds that may be included in an amphiphilic layer include, but are not limited to, phospholipids, aminolipids, cationic amphiphilic drugs, and sphingolipids. Bilayer membranes of such nanoparticles are typically formed by amphiphilic molecules, such as lipids of synthetic or natural origin that comprise spatially separated hydrophilic and hydrophobic domains. Bilayer membranes of the (lipid) based nanoparticles can also be formed by amphiphilic polymers and surfactants (e.g., polymerosomes, niosomes, etc.). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

In one embodiment, the disclosure is directed to a lipid nanoparticle (LNP) or lipoplex (LPX) formulation (also referred to as lipoplex particle) comprising a lipid component, a nucleic acid encoding an antigen derived from an intracellular bacterium and an iNKT-cell agonist, in particular, a glycolipid antigen such as e.g., α-GalCer, or a functional analogue or derivative thereof.

Hence, in a particular embodiment a nucleic acid, more specific RNA may be present in/complexed to a lipoplex particle. In general, the lipoplex particle contains a lipid, in particular, cationic lipid, and a nucleic acid. Electrostatic interactions between positively charged liposomes and negatively charged nucleic acids results in complexation and spontaneous formation of the particles. Positively charged liposomes may be generally synthesized using a cationic lipid and an additional lipid. In the context of this disclosure, a lipoplex particle is referred to as a nanoparticle. In a particular embodiment, the lipoplex particle as envisaged herein, and as used in the methods of this disclosure, comprises or consists essentially of:
a cationic lipid;
at least one helper lipid, particularly a steroid or sterol;
an iNKT cell agonist; and
a nucleic acid encoding a protein or peptide, in particular, an antigen derived from an intracellular bacterium (an ‘intracellular bacterial antigen’).

In a particular embodiment of the disclosure, the cationic lipid 1,2-dioleoyloxy-3-trimethylammonium propane or “DOTAP” is used to prepare the lipoplex particle. DOTAP can be formulated alone or can optionally be combined with a neutral lipid or other cationic or non-cationic lipid (the so called helper lipid) into a liposomal transfer vehicle or a lipoplex particle. More specifically, the at least one cationic lipid comprises or consists of 1,2-di-0-octadecenyl-3-trimethylammonium propane (DOTMA) and/or 1,2-dioleoyl-3-trimethylammonium-propane (DOTAP). In a further embodiment, the at least one helper lipid comprises or consists of 1,2-di-(9Z-octadecenoyl)-sn-glycero-3-phosphoethanolamine (DOPE), a sterol and/or 1,2-dioleoyl-sn-glycero-3-phosphocholine (DOPC). In an exemplary embodiment, the cationic lipid is DOTAP and the additional lipid is a steroid or sterol, more specifically cholesterol.

In general, the lipid component of the lipoplex particle comprises between 40 mol % and 80 mol % of a helper lipid, in particular, a steroid or sterol, more in particular, cholesterol. In particular, the concentration of the helper lipid is between 55 mol % and 65 mol % of the total lipid amount.

In another embodiment, a nucleic acid, more specific RNA, may be present in/complexed to a lipid nanoparticle (LNP). As known to the person skilled in the art, a lipid nanoparticle typically comprises a selection of different lipid components, such as a cationic and/or ionizable lipid (also sometimes referred to as cationic ionizable lipid), a polymer conjugated lipid such as polyethylene glycol (PEG)-lipids and a (neutral) helper lipid such as a phospholipid. Optionally the nanoparticle further comprises a steroid or sterol such as cholesterol or an analogue thereof.

Cationic lipids have a net positive charges. Generally, cationic lipids possess a lipophilic moiety, such as a sterol, an acyl chain, a diacyl or more acyl chains, and the head group of the lipid typically carries the positive charge. They bind negatively charged nucleic acid by electrostatic interaction. Ionizable lipids may become positively charged lipids that are able to associate with nucleic acids in lipid/LNP-based delivery systems. A positive charge on the LNP also promotes association with the negatively charged cell membrane to enhance cellular uptake. Ionizable lipids have a pKa<7 and have a neutral to mildly cationic charge under physiological pH conditions. The ionizable cationic lipids with primary, secondary, or tertiary amines in the headgroup have been developed for the purposes of encapsulating nucleic acids when the lipid is positively charged at pH values below the pKa (e.g., pH 4), and for almost neutral LNP at physiological pH values. This offers certain benefits over the permanently-charged (cationic) lipids, the foremost of which is that ionizable lipids have been associated with a reduced toxicity and a prolonged blood circulation lifetime.

Examples cationic and ionizable cationic lipids include 4-hydroxybutyl) azanediyl)bis(hexane-6,1-diyl)bis(2-hexyldecanoate; 1,2-dioleoyl-3-trimethylammonium propane (DOTAP); N,N-dimethyl-2,3-dioleyloxypropylamine (DODMA), 1,2-di-0-octadecenyl-3-trimethylammonium propane (DOTMA), 3-(N-(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis, cis-9, 12-oc-tadecadienoxy)propane (CLinDMA), 2-[5′- (cholest-5-en-3-beta-oxy)-3′-oxapentoxy)-3-dimethyl-1-(cis,cis-9′, 12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), 1,1′-((2-(4-(2-((2-(bis(2-hydroxydodecyl) amino)ethyl) (2-hydroxydodecyl) amino)ethyl) piperazin-1-yl)ethyl) azanediyl) bis(dodecan-2-ol) (C12-200), heptatriaconta-6,9,28,31-tetraen-19-yl-4-(dimethylamino) butanoate (DLin-MC3-DM A), N-(2-Hydroxyethyl)-N,N-dimethyl-2,3-bis(tetradecyloxy)-1-propanaminium bromide (DMRIE), [(4- hydroxybutyl)azanediyl]di(hexane-6,1-diyl) bis(2-hexyldecanoate) (ALC-0315), (#)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-!-propanaminium bromide (GAP-DLRIE), (±)-N-(3-aminopropyl)-N, N-dimethyl-2, 3-bis(tetradecyloxy)-1-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy) propan-l-aminium (DOBAQ), 2-({8-[(3b)-cholest-5-en-3-yloxy]octyl}oxy)-N,N-dimethyl-3-[(9Z, 12Z)-octadeca-9, 12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((IS)-1-[(3-aminopropyl) amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-l-amonium bromide (DLRIE), N-(2-aminoethyl)-N,N-dimethyl-2,3-bis(tetradecyloxy) propan-l-aminium bromide (DMORIE), di((Z)-non-2-en-1-yl) 8,8′-((((2 (dimethylamino)ethyl) thio) carbonyl) azanediyl) dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl) oxy) heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}- ethylamino) propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid 02-200); or heptadecan-9-yl 8-((2-hydroxyethyl) (6-oxo-6-(undecyloxy)hexyl)amino)octanoate (SM-102).

In a specific embodiment, the LNP comprises at least one ionizable lipid.

Optimizing the formulation of nucleic acid containing nanoparticles by addition of other hydrophobic moieties, such as e.g., sterols and lipids, in addition to an ionizable/cationic lipid or lipid-like material may enhance particle stability and efficacy of nucleic acid delivery. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In one embodiment, the additional lipid is one of the following neutral lipid components: (1) a phospholipid, (2) a steroid or sterol such as cholesterol or an analogue thereof; or (3) a mixture of a phospholipid and cholesterol or an analogue thereof.

As such, in addition to cationic or ionizable lipids, the lipid nanoparticle nucleic acid formulations of this disclosure typically contain other lipid components, such as phospholipids, a sterol such as cholesterol and/or a polyethylene glycol (PEG)-functionalized lipid (PEG-lipid or PEGylated lipid). As is known by the skilled person, these lipids can improve nanoparticle properties, such as particle stability, delivery efficacy, tolerability and biodistribution.

In a specific embodiment, the lipid component of the LNP as envisaged herein includes one or more PEGylated lipids. A PEGylated lipid is a lipid modified with polyethylene glycol. This may improve the water-solubility and stability of the LNP. A PEGylated lipid may be selected from the non-limiting group consisting of PEGylated phosphatidylethanolamines, PEGylated phosphatidic acids, PEGylated ceramides, PEGylated dialkylamines, PEGylated diacylglycerols, PEGylated dialkylglycerols, and mixtures thereof. Preferably, the PEGylated lipid is selected from the group consisting of DMG-PEG (1,2-Dimyristoyl-rac-glycero-3-methoxypolyethylene glycol), DSPE-PEG, DSG-PEG, or 2-[(polyethylene glycol)-2000]-N,N-ditetradecylacetamide, wherein the molecular weight of PEG ranges from 1-10 kDa. In a further embodiment, the PEGylated lipid is a PEG-OH lipid. A “PEG-OH lipid,” also referred to herein as “hydroxy-PEGylated lipid,” is a PEGylated lipid having one or more hydroxyl (—OH) groups on the lipid and/or on the PEG chain. In one embodiment, a PEG-OH or hydroxy-PEGylated lipid comprises a hydroxyl group at the terminus of the PEG chain.

In a further embodiment, the lipid component of the LNP as envisaged herein includes one or more steroid or sterol, more preferably cholesterol or an analogue thereof. Incorporation of a steroid or a sterol in the LNP may help aggregation of other lipids in the particle. As defined herein, “sterols” are a subgroup of steroids consisting of steroid alcohols. A particularly preferred sterol is cholesterol or an analogue thereof. Suitable cholesterol-based lipids include, for example, DC-Cholesterol 3beta-[N-(N′,N′-dimethylaminoethane)-carbamoyl] cholesterol, 1,4-bis(3-N-oleylamino-propyl) piperazine or ICE. Other examples are C-24 alkyl phytosterols, ergosterol, fecosterol, sitosterol, campersterol, stigmasterol, brassicasterol, tomatidine, ursolic acid and alpha-tocopherol.

In a further embodiment, the lipid component of the LNP as envisaged herein includes one or more phospholipids. Specific phospholipids that can be used include, but are not limited to, phosphatidylcholines, phosphatidylethanolamines, phosphatidylglycerols, phosphatidic acids, phosphatidylserines or sphingomyelin. Such phospholipids include, in particular, diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular, diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In a particular embodiment, the additional (helper) lipid is DSPC.

In one embodiment, the concentration of the cationic or ionizable lipid in the nanoparticle is between 10 mol % and 90 mol %, between 15 mol % and 85 mol %, between 20mol % and 80 mol %, in particular, between 25 mol % and 70 mol %, and more in particular, between 30 mol % and 65 mol % of the total lipid amount in the particle.

In a further embodiment, the molar ratio of the at least one cationic or ionizable lipid to the at least one helper lipid is from about 10:0 to about 1:9, about 4:1 to about 1:2, or about 3:1 to about 1:1.

In particular, the helper lipid, such as a non-cationic lipid and/or neutral lipid, (e.g., one or more phospholipids and/or steroid or sterol) may be absent or may comprise from about 0.5 mol % to about 90 mol %, from about 1 mol % to about 80 mol %, from about 1 mol % to about 70 mol %, from about 1 mol % to about 60 mol %, or from about 1 mol % to about 50 mol %, of the total lipid present in the particle.

As used herein, an “INKT cell agonist” (also referred to herein as a “INKT ligand”) has its general meaning in the art and refers to any derivative or analogue from a (glycol) lipid that is typically presented in a CDId molecule by antigen presenting cells (APCs) and that is recognized by a conserved, semi-invariant αβ T cell receptor expressed by iNKT cells, thereby activating iNKT cells, i.e., promote, in a specific manner, cytokine production by iNKT cells. iNKT agonistic activity of compounds can be determined by methods as provided in e.g., Zajonc et al., 2005, or Watarai et al., 2008 (incorporated herein by reference). In one embodiment, the iNKT cell agonist is a lipid, more specifically a glycolipid, specifically binding CD1d, whereby this agonist-CD1d complex is specifically binding the αβ T cell receptor expressed by iNKT cells. In another specific embodiment, the iNKT cell agonist according to the disclosure is a glycolipid antigen such as a α-galactosylceramide compound. α-Galactosylceramide (α-GalCer; (2S,3S,4R)-1-O-(alpha-D-galactosyl)-N-hexacosanoyl-2-amino-1,3,4-octadecanetriol) having the common name KRN7000, and is an agelasphin derivative. As used herein, the term “α-galactosylceramide compound” or “α-GalCer compound” has its general meaning in the art and includes a functional derivative or analogue. Such derivative or analogue is a glycosphingolipid that contains a galactose carbohydrate attached by an a-linkage to a ceramide lipid that has an acyl and sphingosine chains of variable lengths. A functional analogue or derivative retains the capacity to specifically bind and/or activate iNKT cells. Various publications have described α-GalCer compounds and their synthesis. Functional derivatives or analogues of α-galactosylceramide, are provided in e.g., WO2014001204 (incorporated by reference and specifically referring to the disclosed compounds NU-αGC, PyrC-αGC and OCH), WO201379687, and WO2013162016. Further examples of iNKT cell agonists include: HS44, BbGL-II, threitolceramide, ABX196, PBS-25, PBS-57, α-C-GalCer, OCH, Naphtylureum-α-GalCer or NU-α-GalCer, Alphα-GalCer-6″-(4-pyridyl) carbamate or PyrC-α-GalCer, (3S,4S,5R)-1-(6″-O-(4-pyridinylcarbamoyl)-α-C-D-galacto-pyranosyl)-3-hexacosylamino-nonadecane-4,5-diol, (3S,4S,5R)-1-(6″-O-(4- pyridinylcarbamoyl)-α-C-D-galacto-pyranosyl)-3-hexacosylamino-1-nonadecene-4,5-diol, (3S,4S,5R)-1-(6″-naphtureido-6″-deoxy-α-C-D-galacto-pyranosyl)-3-hexacosylamino-nonadecane-4,5-diol, (3S,4S,5R)-1-(6″-naphtureido-6″-deoxy-α-C-D-galacto-pyranosyl)-3-hexacosylamino-1-nonadecene-4,5-diol, α-1C-GalCer, and 7DW8-5. α-GalCer compounds can be chemically synthesized by methods known to the skilled person. In a particular embodiment of this disclosure, the α-GalCer compound is incorporated in the nanoparticle provided herein, and more specific in the lipid component of the nanoparticle. In an even more specific embodiment, the α-GalCer compound is α-galactosylceramide. The phrases “activate iNKT cells” or “induce iNKT immune response” have similar meanings and refer, for instance, to the observed induction of cytokine production, such as IFN-γ in iNKT cells by α-GalCer compound. Analysis of the activation of iNKT cells can be performed by the methods provided herein or by flow cytometry using CD1d tetramers loaded with αGalCer or derivates such as PBS-57.

The concentration of the iNKT cell agonist, in particular, the α-GalCer compound, more in particular, α-GalCer, in the nanoparticle is between 0.0015 mol % and 5 mol % of the total lipid amount, in particular, in between 0.0015 mol % and 1 mol %, more in particular, between 0.0015 mol % and 0.5 mol %, even more in particular, between 0.0015 mol % and 0.25 mol %.

In a particular embodiment, the LNP as envisaged herein, and as used in the methods of this disclosure, comprises or consists essentially of:
a cationic or ionizable lipid, in particular, an ionizable lipid;
at least one PEGylated lipid;
at least one helper lipid, particularly a steroid or sterol, and, optionally, one other (neutral) helper lipid such as e.g., a phospholipid;
an iNKT cell agonist, in particular, a α-galactosylceramide compound; and
a nucleic acid encoding a protein or peptide, in particular, an antigen derived from an intracellular bacterium.

The nanoparticle of the disclosure may in further embodiments be customized in terms of size, surface charge and attachment of any targeting moieties such as e.g., antibodies, (poly) peptides, folate, carbohydrates (such as mannose, galactose or GalNAc), haloperidol, anisamide, and cardiac glycosides or the like. Moreover, the nanoparticle can further comprise a pan HLA DR-binding epitope (PADRE). Furthermore, the nanoparticle surface can be modified with poly(ethylene glycol) (PEG) or functionally related polymers or moieties that are able to maintain nanoparticle colloidal stability, reduce nonspecific interactions and recognition by the immune system.

### Methods of Preparation

Preparation of the nanoparticles of the disclosure can be by any method known to the skilled person, such as via ethanol dilution, lipid film hydration, or by the use of microfluidic devices. An exemplary method of preparing the RNA loaded lipoplex nanoparticles of this disclosure include the following steps: (1) dissolve appropriate amounts of lipids in chloroform, (2) add an appropriate amount of the iNKT agonist, (3) evaporate the chloroform and rehydrate the resulting lipids in a buffer, (4) reduce the size of the resulting lipid particles through sonication or extrusion, (5) mix with mRNA.

In addition, the RNA loaded lipid nanoparticles as provided herein may be prepared by (1) dissolve appropriate amounts of lipids in ethanol, (2) add an appropriate amount of the iNKT agonist, (3) mixing the solution of the lipids in ethanol into water or a suitable aqueous phase. In some embodiments, the aqueous phase has an acidic pH. In some embodiments, the aqueous phase comprises sodium acetate, e.g., in an amount of about 25 mM. (4) Dialysis of formed mRNA loaded LNPs in aqueous buffer with a neutral pH. In some embodiments, the aqueous phase comprises tromethamine, e.g., in an amount of about 20 mM. (5) Optionally, a cryoprotectants, such as sucrose, e.g., in an amount of about 12.5 w/v %, can be added to the resulted mRNA loaded LNPs.

The agent such as the nucleic acids provided herein may be encapsulated by the nanoparticle or it may be attached to a surface or surfaces thereof to form a conjugate. Suitable methods for encapsulating agents inside nanoparticles are known to the skilled person and comprise electrostatic complexation, covalent coupling, hydrophobic interactions, passive loading, remote loading, salting-out, nanoprecipitation, emulsion-diffusion, solvent-evaporation, spray drying and emulsion polymerization. Typically such methods may be adapted depending upon the materials used to make the nanoparticles and the chosen agent, which adaptation will be within the remit of the skilled person.

In one embodiment, the disclosure relates to a cationic and/or lipid-based nanoparticle in which both nucleic acid, such as RNA, and an adjuvant, such as an iNKT cell agonist provided herein, can be complexed. As used herein, the term “complexed” includes the conjugation, encapsulation, attachment or coupling of the adjuvant with or in the nanoparticle. The term “admixed” refers to the adjuvant that is dissolved, dispersed, or suspended in the nanoparticle. The iNKT cell agonist is associated with, covalently coupled to, or incorporated/encapsulated in the nanoparticle by methods well known to the person skilled in the art or by the method as provided herein. As an example, the iNKT cell agonist can be incorporated in the aqueous core and/or the lipid membrane of lipid-based nanoparticles as provided herein, the iNKT cell agonist can be part of a lipidic or polymeric micelle formulation, or the iNKT cell agonist can be applied in polymeric nanoparticles such as polymer conjugates, polymer matrix nanoparticles and solid polymer nanoparticles.

The amount of nucleic acid, and in particular, RNA, in a nanoparticle composition may depend on the size, sequence, and other characteristics of the nucleic acid. The amount of nucleic acid in a nanoparticle composition may also depend on the size, composition, desired target, and other characteristics of the nanoparticle composition. The relative amounts of nucleic acid and other elements (e.g., lipids) may also vary. In one embodiment, the wt/wt ratio of the lipid component to a nucleic acid in a nanoparticle composition may be from about 1:1 to about 100:1. For example, the wt/wt ratio of the lipid component to a nucleic acid may be from about 10:1 to about 50:1. The amount of nucleic acid such as e.g., RNA in a nanoparticle composition may, for example, be measured using a fluorescence-based RNA-quantitation assay. In some embodiments, the one or more nucleic acids, lipids, and amounts thereof may be selected to provide a specific N:P ratio. The N:P ratio of the composition refers to the molar ratio of nitrogen atoms in one or more lipids to the number of phosphate groups in a nucleic acid, more specific mRNA. It is correlated to the charge ratio, as the nitrogen atoms (depending on the pH) are usually positively charged and the phosphate groups are negatively charged. The one or more nucleic acid/mRNA, lipids, and amounts thereof may be selected to provide an N:P ratio from about 1:2 to about 12, such as 1:2, 1:1, 2:1, 3:1, 4:1, 5:1, and 6:1, and in particular, from about 3:1 to 6:1.

### Composition/Formulation

In a further aspect, the disclosure provides a (pharmaceutical) composition, formulation or delivery system comprising the nanoparticle as provided herein, i.e., containing the genetic material such as the nucleic acid, in particular, mRNA, and the iNKT cell agonist, as provided herein and one or more of a pharmaceutically acceptable excipient, carrier and/or diluent. In one embodiment, the composition is a vaccine. The disclosure also provides the uses as disclosed herein of the composition.

In one embodiment, a pharmaceutical composition described herein is an immunogenic composition for inducing an immune response against an infectious agent, more specifically an intracellular bacterium. As such, the nanoparticles and compositions of this disclosure are ideally suited as effective vaccines, therapies or applications as provides herein against a variety of intracellular bacteria, either obligate or facultative anaerobe, such as Listeria sp., Mycobacterium sp., Legionella sp., Salmonella sp., Chlamydia sp., Rickettsia sp., Brucella sp., Staphylococcus sp., Streptococcus sp., Enterococcus sp., Shigella sp., Coxiella sp., Helicobacter sp., Campylobacter sp., Ehrlichia sp., Yersinia sp., Borrelia sp., Rhodococcus sp., Bartonella sp., Acinetobacter sp., Escherichia sp., Clostridium sp. and Veillonella sp. In a particular embodiment, the infections treated are with obligate or facultative anaerobic Gram-positive bacteria, e.g., selected from the group consisting of Listeria sp., Mycobacterium sp., Enterococcus sp., Streptococcus sp., Staphylococcus sp.,, Clostridium sp. and Rhodococcus sp. In a another embodiment, the infections treated are with obligate or facultative anaerobic Gram-negative bacteria, e.g., selected from the group consisting of Chlamydia sp., Legionella sp., Salmonella sp., Escherichia sp., Yersina sp., Rickettsia sp., Brucella sp., Shigella sp., Coxiella sp., Helicobacter sp., Campylobacter sp., Ehrlichia sp., Bartonella sp., Acinetobacter sp., Veillonella sp., and Borrelia sp. Exemplary species to be treated include Listeria monocytogenes, Mycobacterium bovis, Mycobacterium tuberculosis, Mycobacterium leprae, Mycobacterium Ulcerans, Legionella pneumophila, Salmonella typhimurium, Salmonella enteritidis, Chlamydia pneumonia, Chlamydia trachomatis, Chlamydia psittaci, Neisseria meningitides, Rickettsia rickettsia, Rickettsia typhi, Brucella melitensis, Staphylococcus aureus, Streptococcus pyogenes, Enterococcus faecalis, Shigella dysenteriae, Coxiella burnetii, Helicobacter pylori, Campylobacter jejuni, Ehrlichia chaffeensis, Ehrlichia phagocytophila, Burkholderia pseudomallei, Yersinia pestis, Borrellia Burgdorferi, Borrellia Mayonii, Rhodococcus equi, Bartonella henselae, Acinetobacter baumannii, Escherichia coli and Clostridium botulinum, Clostridium difficile, Clostridium perfringens and Clostridium tetani.

By “immunogenic” is meant the capacity to provoke an immune response in a subject against the pathogen/infectious agent. This disclosure accordingly provides compositions for use in eliciting an immune response that may be utilized as a prophylactic or therapeutic vaccine, in particular, against an intracellular bacterium. The immune response can be an adaptive immune response mediated by cytotoxic CD8 T-cells, helper CD4 T-cells and/or a humoral (B-cell) immune response resulting in the production of antibodies against the bacterial antigen. More specific, by “eliciting or inducing an adaptive immune response” is meant that an antigen stimulates synthesis of specific antigen-specific antibodies and/or cellular proliferation as measured by, for example, the flow cytometric detection of T-cells and enzyme-linked immunosorbent assays to measure antibodies. In this disclosure, it was found that an iNKT agonist can be utilized to increase the capacity of mRNA vaccines to induce adaptive immune responses. In addition, this disclosure also provides a solution to obtain empowered and broadened innate immune responses induced by mRNA vaccines that can cooperate with adaptive components to protect the host against bacterial infections. In this disclosure, innate immune responses are specifically driven by the cellular proliferation and activation of iNKT cells, which are known to be capable of exerting direct cytotoxic functions in several bacterial infections. Moreover, INKT cells participate in the response against intracellular bacteria by mediating the activation of other innate immune cell types through direct cellular interactions and the prompt release of a variety of cytokines, for example, the activation of dendritic cells (i.e., mediated by CD1d recognition and CD40-CD40 ligand interactions), bystander activation of NK cells and macrophages (IFN- [symbol] ) and recruitment and activation of neutrophils (IL-17 and IL-22).

In one embodiment, administering the nanoparticle, composition or vaccine of the disclosure elicits an immune response that results in a reduction in the load of the infectious agent of at least about 30%, 40%, 45% or 50% in a subject in relation to a non-vaccinated control subject. Preferably, the level of the decrease is about 55%, more preferably about 60% and most preferably, about 90%, 95% or greater. As presented in the examples, the bacterial load in a specific organ (or cel suspension derived thereof) of a vaccinated vs. non-vaccinated subject e.g., spleen or liver can be assessed by counting colony-forming units (CFU) after serial dilution and replating. Hence the immune response confers some beneficial, protective effect to the subject against a challenge with the infectious agent. More preferably, the immune response prevents the onset of or ameliorates at least one symptom of a disease associated with the infectious agent, or reduces the severity of at least one symptom of a disease associated with the infectious agent upon subsequent challenge. As such, the nanoparticles or composition of this disclosure prevent progression to active disease and ultimately break the cycle of bacterial transmission.

Given the beneficial medical properties, this disclosure relates, according to another aspect, to the nanoparticles provided herein or the pharmaceutical composition comprising them, for use as a human or veterinary medicine. In particular, the composition is an immunogenic composition, more particular a vaccine.

In another embodiment, nucleic acid, in particular, RNA, disclosed herein may be administered in a pharmaceutical composition that may comprise a pharmaceutically acceptable carrier and may optionally comprise one or more stabilizers etc.

The pharmaceutical compositions according to this disclosure are generally applied in a “pharmaceutically effective amount” and in “a pharmaceutically acceptable preparation.”

The term “pharmaceutically acceptable” refers to the non-toxicity of a material that does not interact with the action of the active component of the pharmaceutical composition. The term “pharmaceutically effective amount” or “therapeutically effective amount” refers to the amount that achieves a desired reaction or a desired effect alone or together with further doses. In the case of the treatment of a particular disease, the desired reaction preferably relates to inhibition of the course of the disease or pathology. This comprises slowing down the progress of the disease and, in particular, interrupting or reversing the progress of the disease. The desired reaction in a treatment of a disease may also be delay of the onset or a prevention of the onset of the disease or the condition. An effective amount of the compositions described herein will depend on the infectious agent, condition to be treated, the severeness of the infection and/or disease, the individual parameters of the subject, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In a further embodiment, a pharmaceutical composition is for therapeutic or prophylactic treatments of a bacterial infection, in particular, an infection with an obligate or facultative intracellular bacterium. A “prophylactic action” prevents the outbreak of a disease or infection with a bacterium after ingress of individual representatives in such a way that subsequent spread thereof is greatly reduced or they are even completely deactivated. A “therapeutically relevant action” frees from one or more disease symptoms or results in the partial or complete reversal of one or more physiological or biochemical parameters that are associated with or causally involved in the disease or pathological change, into the normal state. The respective dose or dose range for the administration of the nanoparticles according to the disclosure is sufficiently large to achieve the desired prophylactic or therapeutic effect of induction of an immune response. In addition, the composition can be used as “adjuvant therapy” given in addition to a primary or initial therapy to maximize its effectiveness in a curative setting, or as a “maintenance” or “consolidative” therapy subsequent to and initial therapy to maximize disease control and delay disease recurrence.

In one embodiment, a pharmaceutical composition disclosed herein may contain salts, buffers, preservatives, and optionally other therapeutic agents. Suitable preservatives for use in a pharmaceutical compositions of the present disclosure include, without limitation, benzalkonium chloride, chlorobutanol, paraben and thimerosal. The term “excipient” as used herein refers to a substance that may be present in a pharmaceutical composition of the present disclosure but is not an active ingredient. Examples of excipients, include without limitation, carriers, binders, sugars, cryoprotectants, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component that may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline. Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice. The pharmaceutically acceptable excipient may be a solid (e.g., calcium phosphate, magnesium stearate, talc, sugars, lactose, dextrin, starch, gelatin, cellulose, polyvinylpyrrolidine, low melting waxes and ion exchange resins), a gel or a liquid. Suitable examples of liquid excipients for oral and parenteral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the excipient can also be an oily ester such as ethyl oleate and isopropyl myristate. Sterile liquid excipients are useful in sterile liquid form compositions for parenteral administration. Liquid pharmaceutical compositions, which are sterile solutions or suspensions, can be utilized by, for example, subcutaneous, intranodular, intrathecal, epidural, intraperitoneal, intravenous and intramuscular injection. In one embodiment, the composition is lyophilized.

In order to support the medical effect, i.e., in particular, the immune response, the pharmaceutical composition may, in an embodiment of the disclosure, also comprise further active compounds, where simultaneous or successive administration is conceivable. As such, the uses and methods disclosed herein can also include the use of a nanoparticle or composition as described herein together with one or more additional (therapeutic) agents for the treatment of disease conditions. The combination of active ingredients may be: (1) incorporated in the present nanoparticle as such, e.g., as a further mRNA; (2) co-formulated and administered or delivered simultaneously in a combined formulation; (3) delivered (e.g., by alternation, subsequently or in parallel) as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used. In some cases, a compound disclosed herein is administered and/or formulated with a second therapeutic.

As used herein, the term “subject” refers to humans or any animal, including, but not limited to, mammals, non-human primates, rodents, and the like, to which the nanoparticles, compositions and methods of this disclosure are administered. Typically, the terms “subject” and “patient” are used interchangeably herein in reference to a human or animal subject.

### Administration

The nanoparticle or pharmaceutical composition described herein may be administered intravenously, intraarterially, subcutaneously, intradermally or intramuscularly. In one embodiment, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the nanoparticle or pharmaceutical composition is formulated for intramuscular or subcutaneous administration. In another embodiment, the pharmaceutical composition is formulated for systemic administration, e.g., for intravenous administration.

One route of administration is intravenous administration, and is, in particular, envisaged for the lipoplex particle as described herein. Another route of administration is intramuscular injection, and is, in particular, envisaged for the LNP particle as described herein. Additionally, the nanoparticle or composition comprising the nanoparticle may be delivered to a patient using any standard route of administration, including oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

Alternately, the compositions of the disclosure may be administered in a local rather than systemic manner, for example, via injection of the pharmaceutical composition directly into a targeted tissue, e.g., in a sustained release formulation. Local delivery can be affected in various ways, depending on the tissue to be targeted. For example, aerosols containing compositions of this disclosure can be inhaled (for nasal, tracheal, or bronchial delivery); compositions of this disclosure can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of this disclosure complexed with therapeutic molecules or ligands can even be surgically administered, for example, in association with a polymer or other structure or substance that can allow the compositions to diffuse from the site of implantation to surrounding cells. Alternatively, they can be applied surgically without the use of polymers or supports.

Administration may take the form of single dose administration, or the nanoparticles or composition as disclosed herein can be administered over a period of time, either in divided doses or in a continuous-release formulation or administration method (e.g., a pump). The amounts of composition administered and the route of administration chosen should be selected to permit efficacious treatment of the disease condition. In one embodiment, the nanoparticle or composition of this disclosure are administered to a subject once a day, twice a day, daily or every other day. In a preferred embodiment, the nanoparticles or compositions of this disclosure are administered to a subject twice a week, once a week, every ten days, every two weeks, every three weeks, or more particular every four weeks, once a month, every six weeks, every eight weeks, every other month, every three months, every four months, every six months, every eight months, every nine months or annually.

The nanoparticles or compositions of the disclosure may be used in a monotherapy for treating, ameliorating, reducing, reducing the risk of or preventing a disease, in particular, an infection, even more in particular, an infection with an intracellular bacterium. Alternatively, the nanoparticles or compositions may be used as an adjunct to, or in combination with, known therapies that may be used for treating, ameliorating, reducing the risk of or preventing a disease.

All of the features described herein (including any accompanying claims, abstract and drawings), and/or all of the steps of any method or process so disclosed, may be combined with any of the above aspects in any combination, except combinations where at least some of such features and/or steps are mutually exclusive. The disclosure will be further described by the following figures, tables and examples, which are not intended to limit the scope of protection as defined in the claims.

### EXAMPLES

### Materials and Methods

### Animals and Listeria Culture

The animals were housed in a temperature-(21° C.) and humidity-(60%) controlled environment with 12 h light/dark cycles; food and water were provided ad libitum. All experiments were done under conditions specified by law and authorized by the Institutional Ethical Committee on Experimental Animals. Listeria monocytogenes (EGD BUG600 strain; Murray et al. 1926; Thery et al. 2021) was grown in brain heart infusion (BHI) medium at 37° C. Bacteria were cultured overnight and then sub cultured 1:10 in BHI medium for 2 h at 37° C. Bacteria were washed three times in PBS and resuspended in PBS at 7.5×10⁵ bacteria per 100 μl or further diluted to lower bacteria doses before administration.

### mRNA Constructs

The pGEM4z-OVA-A64 plasmid DNA and pGEM-4z Sig-tOVA-LAMP1plasmid were kindly provided by Prof. Dr. K. Breckpot (VUB, Vrije Universiteit Brussel, Belgium).

For the pGEM-4z SP-tOVA-MITD, the SP and MITD sequence were determined through in silico PCR using the following primers: sec sense, 5′-aag ctt agc ggc cgc acc atg cgg gtc acg geg ccc cga acc-3′ (SEQ ID NO: 3); sec antisense, 5′-ctg cag gga gcc ggc cca ggt ctc ggt cag-3′ (SEQ ID NO:4);. Corresponding sequences of the HLA-B27 Alpha chain protein were attached on the 5′ and 3′ end, respectively, onto truncated ovalbumin (tOVA) and ordered as a gene gBlocks™M Gene Fragment (integrated DNA technologies (IDT) genes). Insert was codon optimized for mouse using the IDT codon optimization tool, (Integrated DNA Technologies, Inc.), cloned in-frame using Gibson Assembly between restriction sites Ncol and EcoRI and the final plasmid product was confirmed by sequencing.

The gene sequence of the OppA lmon_0149 Listeria gene (Uniprot Acc. No. A0A3QONAQ5_LISMG) was cloned into a pGEM4z-plasmid vector (Promega) containing a T7 promoter, 5′ and 3′ UTR of human β globulin, and a poly(A) tail by Genscript. This Listeria protein was identified and prioritized as a relevant (new) MHC-I presented Listeria antigen via mass spectrometry immunopeptidomics by the research group of Prof. Francis Impens (VIB). The OppA lmon_0149 Listeria gene was retrieved from the Listeriomics platform (Becavin et al. 2017), codon optimized for mouse using the IDT codon optimization tool, (Integrated DNA Technologies, Inc.) and the final plasmid product was confirmed by sequencing.

The ESAT-6 plasmid was cloned from the pLMCT-eGFP vector provided by Prof. Dr. K. Breckpot (VUB, Vrije Universiteit Brussel, Belgium) (de Mey, 2022). This plasmid contains the eGFP cDNA, flanked by the 5′-and 3′-untranslated regions of the Xenopus laevis β-globin gene and a 140 A long poly-A tail. A unique NcoI site at the 5′ end of the eGFP cDNA and unique XhoI site at the 3′ end, were used to construct the SP-6 kDa early secretory antigenic target (ESAT-6)-MITD plasmid. The gene sequence of the ESAT-6 Mycobacterium tuberculosis H37Rv gene (Uniprot Acc. No. P9WNK7 ESXA_MYCTU), flanked by the SP and MITD sequence, was ordered as a gene gBlock™ Gene Fragment (integrated DNA technologies (IDT) genes). Insert was codon optimized for mouse using the IDT codon optimization tool, (Integrated DNA Technologies, Inc.), cloned in-frame using Gibson Assembly between restriction sites NcoI and XhoI and the final plasmid product was confirmed by sequencing.

The plasmids were linearized using the appropriate restriction enzymes and purified using a QIAquick PCR purification kit (Qiagen, Venlo, The Netherlands). Linearized plasmids were used as templates for the in vitro transcription reaction using the T7 MegaScript kit, including an Anti-Reverse Cap Analog (ARCA, Trilink BioTechnologies) or CleanCap® Reagent AG (Trilink BioTechnologies) for the SP-ESAT-6-MITD plasmid, and chemically modified N1-methylpseudouridine-5′-triphosphate (Trilink BioTechnologies) instead of the normal nucleotide, uridine. The resulting capped mRNAs were purified by DNase I digestion, precipitated with LiCl and washed with 70% ethanol. All mRNAs were analyzed by agarose gel electrophoresis and concentrations were determined by measuring the absorbance at 260 nm. mRNAs were stored in small aliquots at-80° C. at a concentration of 1 μg/μL.

### Lipids

DOTAP (1,2-dioleoyl-3-trimethylammonium-propane), cholesterol, DSPC (1,2-distearoyl-sn-glycero-3-phosphocholine), DMG-PEG 2000 (1,2-Dimyristoyl-rac-glycero-3-methoxypolyethylene glycol-2000) and αGC (Alpha-Galactosyl Ceramide) were purchased from Avanti Polar Lipids (Alabaster, USA). C12-200 was purchased from Corden Pharma (Plankstadt, Germany).

### Lipid Formulations

### LNP Formulation

Ionizable C12-200 LNPs containing mRNA were prepared as previously described (Kulkarni et al., 2019). Briefly, lipid components (C12-200, DSPC, cholesterol, PEG-DMG) were dissolved in ethanol at a component molar ratio of ˜50/10/38.5/1.5 with a final concentration of 10 mM total lipid. For the LNP formulations with αGC, 0.02 mol % of the total lipid amount was replaced by αGC. mRNA was dissolved in 25 mM sodium acetate buffer at pH 4.0. The organic and aqueous solutions were mixed through a T-junction mixer at a flow ratio of 1:3 (v: v) and a total flow rate of 28 mL min. In Examples 3, 4 and 8, mRNA content was mixed with the lipids dissolved in ethanol to obtain mRNA LNPs at a C12-200: mRNA weight ratio of 10:1. The resting suspension was dialyzed against a 1000-fold volume of PBS buffer for 4 hours. In Examples 1, 5-7, mRNA LNPs were produced by a slightly different protocol, in order to preserve critical particle characteristics and functionality upon freezing and storage at −80° C. More specifically, the C12-200: mRNA weight ratio was increased from 10:1 to 20:1, dialysis buffer was changed to Tris buffer (20 mM), and sucrose was added as cryoprotectants before freezing at −80° C.

The ionizable LNP formulations were subjected to a size and zeta potential quality control using a Malvern Zetasizer nano-ZS (Malvern Instruments Ltd., Worcestershire, UK). The Quant-iT RiboGreen RNA Assay was used to determine mRNA encapsulation and concentration in lipid particle formulations according to manufacturer's protocols (ThermoFisher, Waltham, MA). In order to release and detect the encapsulated mRNA content, mRNA particles were diluted in TE buffer containing 1% (v/v) Triton X-100 (Sigma) and incubated for 10 min at 37° C., while the free (not-encapsulated) mRNA content was directly measured after particle dilution in TE buffer.

### Lipoplex Formulation

Cationic liposomes of DOTAP-cholesterol (2:3 molar ratio) were prepared by the thin-film hydration method (Verbeke et al. 2019). The appropriate amounts of lipids, dissolved in chloroform were transferred into a round-bottom flask. To co-formulate αGC, 0.015 mol % of the total lipid amount was replaced by αGC. The chloroform was evaporated under nitrogen, after which the lipid film was rehydrated in HEPES buffer (20 mM, pH 7.4, Sigma-Aldrich) to obtain a final lipid concentration of 12.5 mM. The resulting cationic liposomes were sonicated in a bath sonicator (Branson Ultrasonics, Dansbury, CT, USA). Then, they were mixed with mRNA to obtain mRNA lipoplexes at a cationic lipid-to-mRNA (N/P) ratio of 3, in a final formulation of an isotonic HEPES buffer containing 5% glucose (Sigma-Aldrich).

### Prime-Boost Vaccination and Listeria Infection

### Example 1

Female C57BL/6 mice (Charles River Laboratories, France) at 7 weeks of age were vaccinated intramuscularly with 2 μg N1-methyl pseudo uridine (m1ψ) modified mRNA encoding a newly discovered listeria protein antigen (OppA lmon_0149) formulated in C12-200iLNPs alone, or co-formulated with αGC adjuvant, or as controls with 2 μg of OVA-mRNA (m1ψ) formulated in C12-200 iLNPs alone, or co-formulated with αGC adjuvant, a sublethal dose of Listeria monocytogenes (5×10⁴ bacteria in 100 μl PBS), or PBS (100μl) at day 0 and day 14 of the experiment. On day 28, the mice were infected intravenously by tail vein injection with 7.5×10⁵ bacteria per animal. Mice were sacrificed 72 h following infection. After tissue homogenization, Colony Forming Units (CFUs) per organ (liver or spleen) were enumerated by serial dilutions in sterile saline and plating on BHI agar.

### Example 2

Female C57BL/6 mice (Charles River Laboratories, France) at 7 weeks of age were vaccinated intravenously with 10 μg NI-methyl pseudo uridine (m1ψ) modified mRNA encoding a newly discovered listeria protein antigen (OppA lmon_0149) formulated in DOTAP-cholesterol lipoplexes co-formulated with αGC adjuvant, or as controls with a sublethal dose of Listeria monocytogenes (1×10⁴ bacteria in 100 μl PBS), or PBS (100μl) at day 0 and day 14 of the experiment. On day 28, the mice were infected intravenously by tail vein injection with 7.5×10⁵ bacteria per animal. Mice were sacrificed 72 h following infection. After tissue homogenization, Colony Forming Units (CFUs) per organ (liver or spleen) were enumerated by serial dilutions in sterile saline and plating on BHI agar.

### Immunization Studies with OVA-mRNA LNPs

Before administration of mRNA lipid nanoparticles, female C57BL/6 mice (Envigo, Gannat, France) at 7 weeks of age were anesthetized in a ventilated anesthesia chamber with 3% isoflurane in oxygen. Nanoparticles with the indicated cargo diluted in PBS buffer were injected either intravenously (IV), intramuscularly (IM) or subcutaneously (SC). Mice received a single dose or 2-dose with a two weeks interval of mRNA. The administered mRNA doses are specified in the examples.

### Flow Cytometric Analysis of T Cell-and iNKT Cell Responses

At different time points after immunization, mice were sacrificed and spleen and inguinal lymph nodes were harvested and processed into single cell suspensions. Spleen and inguinal lymph nodes were gently pushed through a 40 μm cell strainer After single cell suspensions were obtained, a red blood cell lysis step was performed using a red blood cell lysis buffer (Biolegend).

Single-cell suspensions were stained with either fixable viability dye eFluor 450 (Thermo Scientific) or Zombie Yellow (Biolegend, San Diego, CA, USA) according to the manufacturer's instructions to exclude dead cells from analysis, incubated with Fc block (CD16/32) to block nonspecific FcR binding (BD Biosciences, Erembodegem, Belgium), and surface stained with the indicated antibodies and tetramers. T cells were stained with monoclonal antibodies, including CD3e-PE (145-2C11), CD4-FITC (GK1.5), CD8α-APC (53-6.7) (All BD Biosciences). BV450-conjugated H-2Kb/SIINFEKL tetramer (OVA-tetramer) and PE-conjugated I-A(b)/4-17 QQWNFAGIEAAASA tetramer (ESAT-6 Mtb tetramer) were obtained from the National Institutes of Health (NIH) Tetramer Core Facility, and used according to the facility's guidelines. iNKT cells were stained with TCRβ-APC (H57-597, Biolegend), and mCD1d PBS-57 PE tetramer obtained from the NIH tetramer Core Facility, and PD-1-FITC (RMP1-30B) and CXCR5-SB600 (SPRCL5) from BD Biosciences were used to discriminate the follicular helper NKT cellular subset. After additional washing steps, samples were measured by a MACSQuant 16 flow cytometer and analyzed by FLOWJO® software (BD company). Compensation for spectral overlap was calculated using UltraComp eBeads compensation beads (Thermo-Scientific) stained with individual fluorochrome-conjugated antibodies.

To detect OVA-specific IFN- [symbol] producing T cells by an intracellular cytokine staining, 2×10⁶ spleen cells were transferred in a round bottom 96 well plate (200 μl volume) and ex vivo restimulated with 1 μg/ml of the OVA-derived peptides SIINFEKL (CD8; SEQ ID NO:1) and ISQAVHAAHAEINEAGR (CD4; SEQ ID NO:2) (both from Eurogentec, Seraing, Belgium) or culture medium (no peptide) as control in the presence of a Protein transport Inhibitor Cocktail of Brefeldin A and Monensin (eBioscience). Following 37° C. incubation for 5 hours, cells were stained for viability and extracellular antigens after blocking Fc binding sites as described before. Cells were then fixed and permeabilized with BDCytoFix/CytoPerm solution (BD), intracellular staining using an IFN- [symbol] -BV421 antibody (XMG1.2 clone, Biolegend) was performed in perm buffer for 30 min at RT.

To analyze cytokine production during recall responses to ESAT-6 after prime-boost vaccination, supernatants were collected from ex vivo splenocytes 24 h post stimulation with recombinant ESAT-6 protein (10 μg/ml, NR-49424, Bei Resources) and screened for 13 T helper cytokines.

### Measurements of Mouse Ovalbumin Binding IgG Antibodies and Cytokine Responses

Whole blood was collected in heparin coated tubes, centrifuged at 14,000 rpm for 5 minutes to separate serum, after which samples were stored at −80° C. Sera from individual mice were collected two weeks after second vaccination (day 28). The samples were 100, 10.000 or 50.000 times diluted before anti-OVA IgG1 and IgG2c titers were quantified with commercial mouse antibody assay kits (Chondrex, Woodinville, WA, USA), according to the manufacturer's instructions. Cytokines were measured in sera by means of multiplex bead arrays (LEGENDplex™ Mouse Inflammation Panel and Mouse Th helper cytokines, Biolegend).

### RESULTS

### Example 1

In this example, it is demonstrated that the inclusion of α-GalCer (αGC) adjuvant can drastically increase the potency of a mRNA-LNP vaccine to achieve antibacterial immunity against infection with Listeria monocytogenes. Listeria monocytogenes is a foodborne intracellular bacterial pathogen capable of causing severe disease in mice and humans (i.e., listeriosis). Listeria monocytogenes is commonly used as a model for intracellular bacterial infections.

Mice were intramuscularly vaccinated twice on day 1 and day 14 with LNPs alone, or a LNP formulation supplemented with αGC, containing m1ψ mRNA encoding the Oppa lmon_0149 protein (mRNA dose of 2 μg/mouse). Ovalbumin-encoding mRNA (OVA) formulations and an additional PBS negative control were also included to elucidate the potential immune-stimulatory effect of the mRNA-LNP formulations without a pathogen-related antigen. Moreover, in this experiment a positive control is included injecting intravenously low amounts of Listeria monocytogenes EGD (5×10⁴ bacteria). These low-dose infections result in an acute listeriosis that is easily overcome by the animals, leading to a protective adaptive immune response indicating the maximum level of protection that could potentially be reached by vaccination.

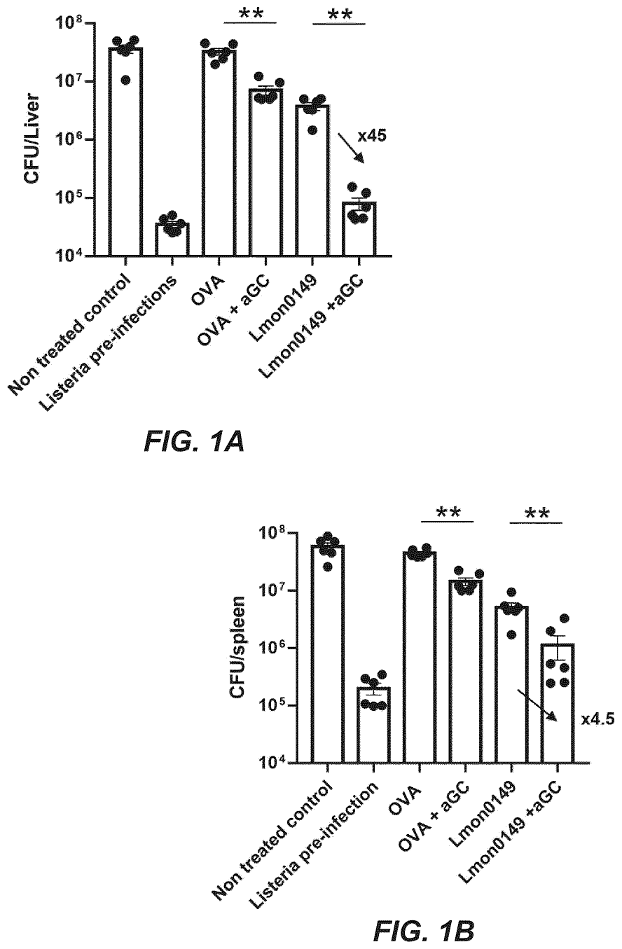

Two weeks after the last vaccination, mice were intravenously challenged with Listeria monocytogenes (7.5×10⁵ bacteria) and three days later, spleen and liver were harvested to determine the infection rate of the mice. FIG. 1 illustrates the bacterial load (colony forming units, CFU) in the liver (panel A) and in the spleen (panel B) of infected mice. Vaccination with lmon_0149 mRNA LNPs resulted in approx. 90% reduction of bacterial load in liver and spleen, whereas the formulation containing αGC remarkably suppressed bacterial levels by 99.7% in liver and 98.1% in spleen, respectively, to CFU levels 45 and 4.5 times lower than for the formulation without αGC. As such, the mRNA-LNP vaccine with αGC could provide almost the same protection as the positive control of the listeria pre-infections (99.9% in liver and 99.6% in spleen). Interestingly, the OVA-mRNA-LNP control did not significantly reduce the bacterial load compared to the PBS control, while an 80.4% in liver and 75.3% in spleen reduction was observed with the OVA formulation containing the αGC adjuvant. This indicates that the activation of NKT cells by the αGC adjuvant also contributes to the protection against Listeria infection in a non-antigen specific manner, thus suggesting that αGC-activated iNKT cells not only promote adaptive immune responses, but also provide additive, or even synergistic protective mechanisms of innate immunity to mRNA vaccines for bacterial infections.

### Example 2

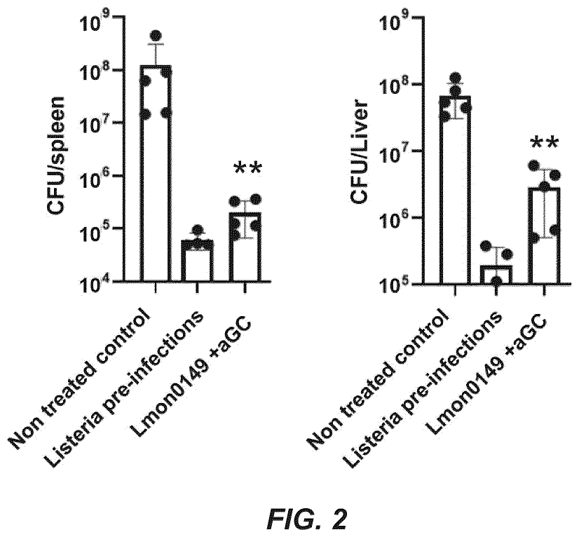

In this example, an immunization study was performed with a lipoplex formulation containing m1ψ lmon_0149 mRNA and the αGC adjuvant that was administered intravenously at a mRNA dose of 10 μg. As evident from FIG. 2, these results show that a cationic liposomal formulation can also be used as carrier for the co-delivery of nucleoside-modified mRNA and aCC adjuvant, resulting in effective protective immunity against infection with Listeria monocytogenes.

### Example 3

In this example, the potency of the αGC adjuvant to empower a m1ψ modified mRNA-LNP vaccine's capacity to induce T cell-and humoral responses was investigated. For this, C57BL/6 mice were immunized twice with mRNA LNPs alone, or a formulation supplemented with αGC (mRNA dose of 10 μg/mouse). The formulations were administered at a two weeks interval, through intravenous (IV), intramuscular (IM) or subcutaneous (SC) administration, where after adaptive immune responses were measured 14 days after the second immunization.

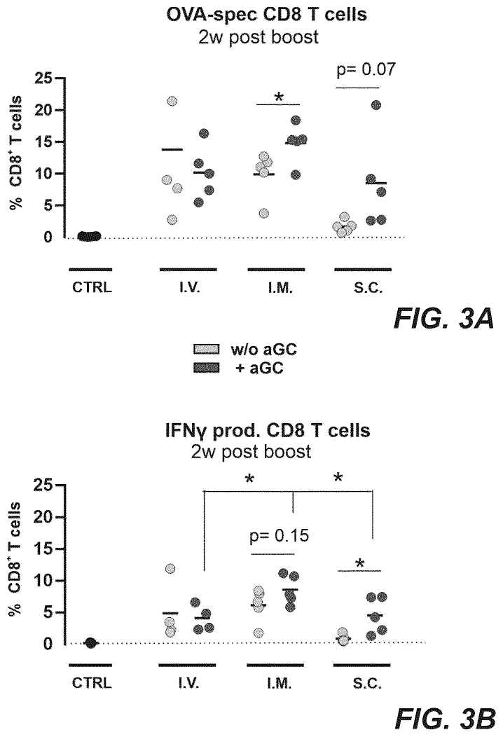

Cytotoxic T lymphocytes (CTLs) were measured in the spleen by an OVA tetramer staining (FIG. 3A). Additionally, splenocytes were re-stimulated with SIINFEKL-peptide and IFN- [symbol] producing cells were detected by performing an intracellular cytokine staining (FIG. 3B). A moderate contribution of αGC to the CTL response was seen for the C12-200 mRNA LNPs when the particles were intramuscularly administered (i.e., 1.5-to-2 fold increase). In particular, for the SC route CTL responses were rather low or undetectable with C12-200 LNPs, but could be strongly increased (i.e., 5-fold) by the inclusion of the αGC adjuvant. In contrast, for the IV route no differences were found between C12-200 mRNA LNPs containing αGC and C12-200 mRNA LNPs alone. It should also be noted that C12-200 mRNA LNPs alone already showed a high potential to induce cellular immunity after IV or IM delivery.

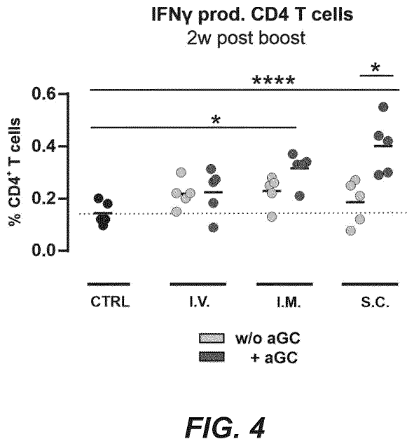

Antigen specific CD4 T cells were also measured in the splenocytes isolated on day 14 after boost vaccination (FIG. 4). Upon pulsing the cells with the OVA-derived MHC-II peptide ISQAVHAAHAEINEAGR (SEQ ID NO: 2), 2-to-3 times higher frequencies of reactive CD4 T cells were detected, when the αGC adjuvant was included, in the mice that were IM and SC vaccinated with C12-200 LNPs. Comparing the different formulations' capacities to induce IgG antibody responses, the αGC adjuvant contributed to higher anti-OVA IgG1 titers (˜3-fold), when the mRNA-LNPs were administered IM, but this was not the case for the other administration routes. For the IgG2c subclass, there were no significant differences found between the C12-200 mRNA LNPs containing αGC and C12-200 mRNA LNPs alone.

Taken together, these findings demonstrate that the αGC adjuvant can increase the potency of mRNA-LNP vaccines to induce cellular-and humoral immunity, and that this, in part, can potentially explain why vaccination with C12-200 mRNA LNPs containing αGC conferred higher protection against listeria infection compared to C12-200 mRNA LNPs alone.

### Example 4

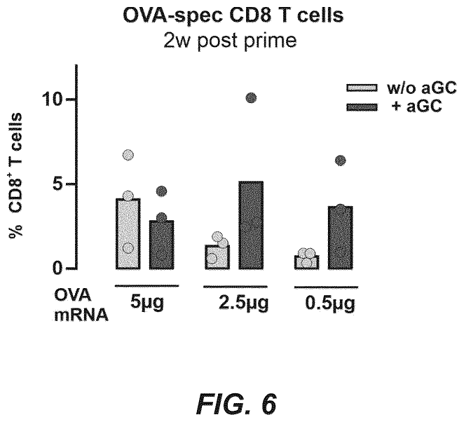

For the IM administration route that was found to be the most effective to induce adaptive immune responses, a dose-reduction study was also performed. Here, C12-200 mRNA LNPs alone, or in the combination with αGC were administered at mRNA doses of 5 μg, 25 μg or 0.5 μg (FIG. 6). Two weeks after a single immunization the frequency of OVA-CTLs in the spleen was measured. As evident from FIG. l , there is a dose-dependent induction of OVA-CTLs in the C12-200 mRNA LNPs group, where the 0.5 μg dose of this formulation barely induced a T cell response. In contrast, robust CTL responses where seen with both the 5 μg, 2.5 μg and 0.5 μg dose of C12-200 LNPs containing αGC. Moreover, nanoparticles with αGC at a dose of 0.5 μg mRNA showed an equal potency to elicit CTL responses than nanoparticles without αGC at a dose of 5 μg mRNA. Importantly, this result indicates a dose-sparing effect of the αGC adjuvant fold for inducing CTL responses, where the dose of mRNA-LNP vaccine can be reduced by at least 10-fold.

### Example 5

In this example, an mRNA construct encoding for the model antigen OVA was optimized with different trafficking sequences to further enhance T cell immunity. More specifically, truncated ovalbumin (tOVA) as model antigen was either flanked by sequences of the signal peptide (SP) and MHC class I trafficking domain (MITD) of the HLA-B27 Alpha chain protein (i.e., SP-tOVA-MITD construct) or by the targeting sequence lysosome-associated membrane protein-1 (i.e., Sig-tOVA-LAMPI construct), as previously described by Kreiter et al. and Bonehill et al., respectively.

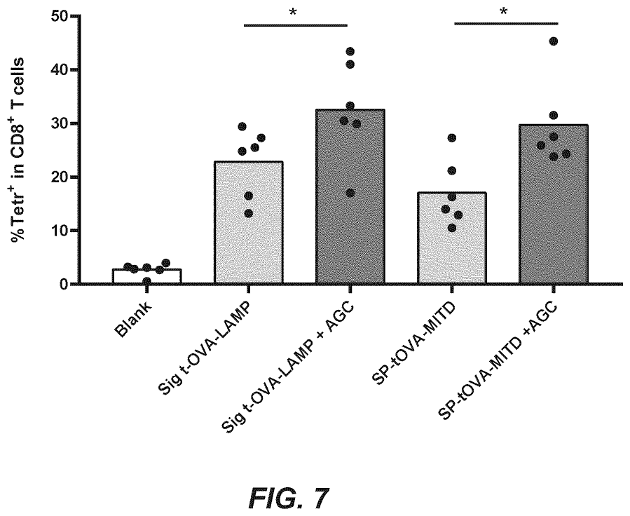

As shown in FIG. 7, upon two immunizations, levels of OVA-specific CD8 T cells were significantly higher when αGC was included in an mRNA-LNP vaccine (mRNA dose of 2 μg), with an increase of 50% for Sig-tOVA-LAMPI mRNA and 70% for SP-tOVA-MITD mRNA.

### Example 6

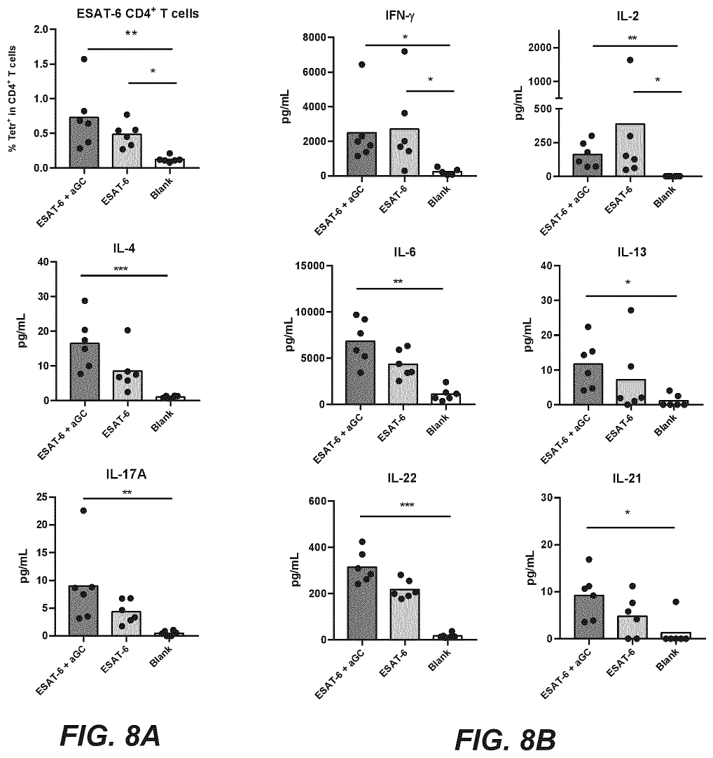

ESAT-6 is a leading vaccine antigen candidate against Mycobacterium tuberculosis, aimed at eliciting ESAT-6-specific CD4 T cells to confer protective immunity. In this example, ESAT-6 was encoded as full-length protein in an mRNA construct optimized with signal peptide (SP) and MHC class I trafficking domain (MITD) using the following nucleic acid sequences: SP: ATG CTT GTA ATG GCA CCT CGG ACT GTA CTT CTC CTC CTC TCC GCC GCC CTC GCA TTG ACC GAA ACC TGG GCT GGC TCT (SEQ ID NO:5), ESAT-6: ACT GAA CAG CAG TGG AAT TTT GCC GGG ATC GAG GCA GCC GCT TCA GCC ATC CAA GGT AAT GTC ACC AGT ATC CAT TCC TTG CTT GAT GAA GGC AAG CAA TCT CTG ACT AAG TTG GCA GCT GCA TGG GGG GGT AGC GGG AGT GAA GCC TAT CAG GGC GTT CAG CAG AAA TGG GAC GCC ACT GCC ACT GAG TTG AAC AAT GCC TTG CAG AAC CTT GCT CGA ACC ATA AGC GAG GCA GGT CAG GCT ATG GCA TCC ACT GAA GGA AAC GTC ACA GGG ATG TTC GCC (SEQ ID NO:6), MITD: ATT GTA GGC ATC GTT GCC GGA CTG GCT GTA CTT GCT GTC GTT GTT ATC GGA GCC GTC GTA GCC GCT GTG ATG TGT AGG CGG AAA TCC AGC GGA GGG AAG GGG GGG TCA TAT TCT CAG GCT GCA TGT TCA GAT TCT GCT CAA GGT AGT GAC GTA AGT TTG ACA GCC TGA (SEQ ID NO:7), formulated in C12-200 LNPs and administered alone, or in the combination with αGC. After two IM vaccinations at an mRNA dose of 2 μg, an equal induction of ESAT-6 specific CD4 T cell responses was measured via tetramer staining (FIG. 8A). However, upon restimulation of splenocytes with ESAT-6 protein, a higher diversity of Th cell responses was seen when αGC was included in the mRNA vaccine, as evident by the detection of similar levels of Th1 cytokine responses (IFN- [symbol] and IL-2), but a higher secretion of Th2 cytokines (IL-4 and IL-6), Th22 cytokines (IL-22, IL-13) and Th17 cytokines (IL-17A, IL-21) (FIG. 8B). Since Th1 CD4 T cells are essential, but not sufficient to confer protection against Mycobacterium tuberculosis, there is growing evidence that mixed CD4 T cell responses may be important for vaccine potency.

### Example 7

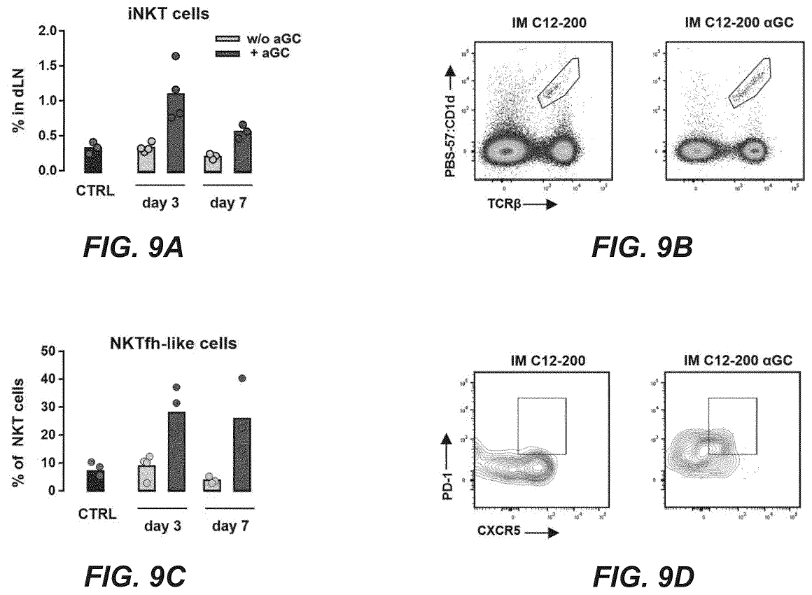

In this example, C12-200 mRNA LNPs alone, or in the combination with αGC were administered intramuscularly at an mRNA dose of 8 μg, and 3 days or one week post-injection iNKT cells were measured in the draining lymph node (dLN) from the injection site. As evident from FIGS. 9A-B, the frequency of iNKT cells increased 2-fold by 72 hours in the dLN of the animals that received the combination of mRNA and the iNKT agonist, and iNKT cell levels remained higher for at least 7 days post-injection. In addition, an upregulation of CXCR5 and PD-1 was detected in a fraction of these iNKT cells, also indicating the generation of follicular helper NKT (NKTfh) cells (FIG. 9C-D).

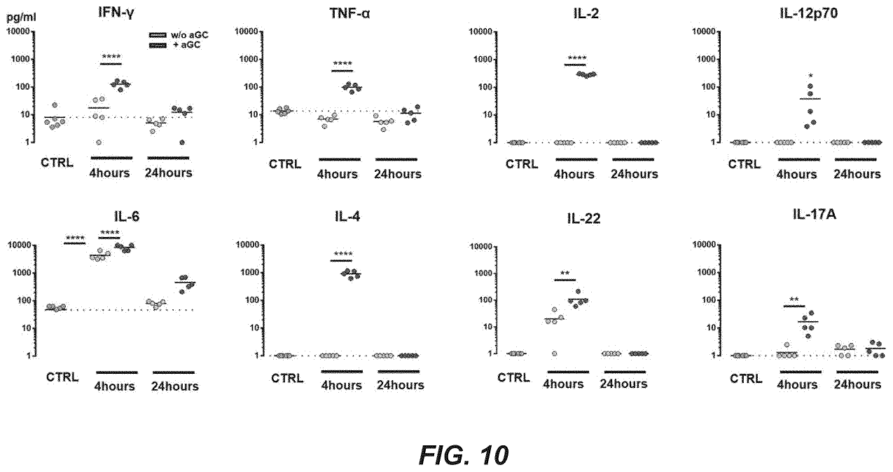

A striking feature of activated NKT cells is that these cells rapidly secrete large amounts of cytokines that in turn can contribute to the activation of NK cells, dendritic cells, macrophages, T cells and B cells. In addition, the production of cytokines by NKT cells, such as IL-17 or IL-22, may have an important role in microbial defense, e.g., by recruiting neutrophils and by inducing the production of antimicrobial peptides. FIG. 10 depicts cytokines measured in collected sera at 4 hours and 24 hours post-injection; while mRNA-LNP formulation alone trigger the production of IL-6 and MCP-1, administration of αGC-adjuvant loaded mRNA LNPs resulted in a highly significant but transient increase of distinct cytokines, including IL-6, IFN- [symbol] , TNF-α, IL-2, IL-12p70, IL-4, IL-22 and IL-17A. These cytokine responses are indicative for an enhanced and broader innate immune activation that can be obtained upon mRNA vaccination against bacterial infections, when the αGC adjuvant is included.

### REFERENCES

Becavin, C. et al. Listeriomics: an Interactive Web Platform for Systems Biology of Listeria. mSystems 2, Host-Microbe Biology March/April 2017 Volume 2 Issue 2 e00186-16.
Bonehill et al. Messenger RNA-Electroporated Dendritic Cells Presenting MAGE-A3 Simultaneously in HLA Class I and Class II. J Immunol (2004) 172 (11): 6649-6657.
De Mey et al. Molecular Therapy: Nucleic Acids Vol. 29, p943, September 2022.
Kreiter et al. Increased Antigen Presentation Efficiency by Coupling Antigens to MHC Class I Trafficking Signals. J Immunol (2008) 180 (1): 309-318.
Kulkarni et al. Fusion-dependent formation of lipid nanoparticles containing macromolecular payloads Nanoscale, 2019, 11, 9023-9031.
E. G. D. Murray, R. A. Webb, M. B. R. Swann A disease of rabbits characterised by a large mononuclear leucocytosis, caused by a hitherto undescribed bacillus Bacterium monocytogenes (n.sp.) The Journal of Pathology and Bacteriology 1926 Volume 29, Issue 4 p. 407-439.
Thery, F., Martina, L., Asselman, C. et al. Ring finger protein 213 assembles into a sensor for ISGylated proteins with antimicrobial activity. Nat Commun 12, 5772 (2021).
Verbeke R, Lentacker I, Breckpot K, Janssens J, Van Calenbergh S, De Smedt SC, Dewitte H. Broadening the Message: A Nanovaccine Co-loaded with Messenger RNA and α-GalCer Induces Antitumor Immunity through Conventional and Natural Killer T Cells. ACS Nano. 2019 Feb. 26;13(2): 1655-1669.
Verbeke R, Lentacker I, De Smedt SC, Dewitte H. The dawn of mRNA vaccines: The COVID-19 case. J Control Release. 2021 May 10;333:511-520.
Qi, H., Shan, P., Wang, Y., Li, P., Wang, K., & Yang, L. (2021). Nanomedicines for the Efficient Treatment of Intracellular Bacteria: The “ART” Principle. Frontiers in chemistry, 9, 775682.
Watarai, H., Nakagawa, R., Omori-Miyake, M. et al. Methods for detection, isolation and culture of mouse and human invariant NKT cells. Nat Protoc 3, 70-78 (2008).
Zajonc D M, Cantu C 3rd, Mattner J, Zhou D, Savage P B, Bendelac A, Wilson I A, Teyton L. Structure and function of a potent agonist for the semi-invariant natural killer T cell receptor. Nat Immunol. 2005 August;6(8): 810-8.

1.-14. (canceled)

15. A method of preventing, reducing, and/or treating an intracellular bacterial pathogen infection in a subject, the method comprising:
administering to the subject a nanoparticle comprising:
an mRNA encoding an antigen of the intracellular bacterium, and
an iNKT cell agonist
so as to treat the subject and prevent, reduce, and/or treat the infection.

16. The method according to claim 15, wherein the nanoparticle further comprises a lipid component.

17. The method according to claim 15, wherein the nanoparticle is a lipoplex particle or a lipid nanoparticle.

18. The method according to claim 15, wherein the nanoparticle comprises:
at least one cationic or ionizable lipid, and at least one helper lipid.

19. The method according to claim 15, wherein the iNKT cell agonist is a glycolipid or an α-GalCer compound.

20. The method according to claim 16, wherein the iNKT cell agonist is incorporated into the lipid component.

21. The method according to claim 16, wherein the concentration of the iNKT cell agonist in the nanoparticle is between about 0.0015 mol % and about 1 mol % of the total lipid amount.

22. The method according to claim 19, wherein the α-GalCer compound is α-galactosylceramide, or a functional derivative or analogue thereof, the functional derivative or functional analogue comprising a glycosphingolipid containing a galactose carbohydrate attached by an α-linkage to a ceramide lipid that has acyl and sphingosine chains of variable lengths.

23. The method according to claim 19, wherein the α-GalCer compound is selected from the group consisting of α-galactosylceramide, HS44, BbGL-II, threitolceramide, ABX196, PBS-25, PBS-57, α-C-GalCer, OCH, naphtylureum-α-GalCer or NU-α-GalCer, α-GalCer-6″-(4-pyridyl) carbamate or PyrC-α-GalCer, (3S,4S,5R)-1-(6″-O-(4-pyridinylcarbamoyl)-a-C-D-galacto-pyranosyl)-3-hexacosylamino-nonadecane-4,5-diol, (3S,4S,5R)-1-(6″-O-(4-pyridinylcarbamoyl)-α-C-D-galacto-pyranosyl)-3-hexacosylamino-1-nonadecene-4,5-diol, (3S,4S,5R)-1-(6″-naphtureido-6″-deoxy-α-C-D-galacto-pyranosyl)-3-hexacosylamino-nonadecane-4,5-diol, (3S,4S,5R)-1-(6″-naphtureido-6″-deoxy-α-C-D-galacto-pyranosyl)-3-hexacosylamino-1-nonadecene-4,5-diol, α-1C-GalCer, and 7DW8-5.

24. The method according to claim 15, wherein the nanoparticle is comprised within a pharmaceutical composition, which pharmaceutical composition further comprises a pharmaceutically acceptable carrier, excipient, and/or diluent.

25. The method according to claim 15, wherein the intracellular bacterial is selected from the group consisting of a Listeria sp., Mycobacterium sp., Legionella sp., Salmonella sp., Chlamydia sp., Rickettsia sp., Brucella sp., Staphylococcus sp., Streptococcus sp., Enterococcus sp., Shigella sp., Coxiella sp., Helicobacter sp., Campylobacter sp., Ehrlichia sp., Yersinia sp., Borrelia sp., Rhodococcus sp., Bartonella sp., Acinetobacter sp., Escherichia sp., and Veillonella sp.

26. The method according to claim 15, wherein the nanoparticle is administered to the subject by intravenous administration, subcutaneous administration, intradermal administration, intramuscular administration, intraperitoneal administration, intranasal administration, or via inhalation.

27. The method according to claim 15, wherein the nanoparticle is capable of inducing a protective immune response against an intracellular bacterial infection in the subject, wherein the immune response is at least 3 to 50 times stronger than an immune response induced in the subject administered a control nanoparticle or composition without an iNKT cell agonist.

28. The method according to claim 16, wherein the nanoparticle is a lipoplex particle or a lipid nanoparticle.

29. The method according to claim 18, wherein the at least one helper lipid is selected from the group consisting of a steroid, a sterol, a phospholipid, a PEGylated lipid, and a combination thereof.

30. The method according to claim 18, wherein the at least one helper lipid is a sterol that is a cholesterol-based lipid.


SEQUENCE LISTING (7 sequences)

SEQ ID NO: 1 - protein, 8 aa, unidentified
SIINFEKL

SEQ ID NO: 2 - protein, 17 aa, unidentified
ISQAVHAAHAEINEAGR

SEQ ID NO: 3 - DNA, 42 bp, synthetic construct
  source 1..42: mol_type = other DNA
aagcttagcggccgcaccatgcgggtcacggcgccccgaacc

SEQ ID NO: 4 - DNA, 30 bp, synthetic construct
  source 1..30: mol_type = other DNA
ctgcagggagccggcccaggtctcggtcag

SEQ ID NO: 5 - DNA, 78 bp, synthetic construct
  source 1..78: mol_type = other DNA
atgcttgtaatggcacctcggactgtacttctcctcctctccgccgccctcgcattgaccgaaacctgggctggctct

SEQ ID NO: 6 - DNA, 282 bp, synthetic construct
  source 1..282: mol_type = other DNA
actgaacagcagtggaattttgccgggatcgaggcagccgcttcagccatccaaggtaatgtcaccagtatccattccttgcttgatgaaggcaagcaatctctgactaagttggcagctgcatgggggggtagcgggagtgaagcctatcagggcgttcagcagaaatgggacgccactgccactgagttgaacaatgccttgcagaaccttgctcgaaccataagcgaggcaggtcaggctatggcatccactgaaggaaacgtcacagggatgttcgcc

SEQ ID NO: 7 - DNA, 168 bp, synthetic construct
  source 1..168: mol_type = other DNA
attgtaggcatcgttgccggactggctgtacttgctgtcgttgttatcggagccgtcgtagccgctgtgatgtgtaggcggaaatccagcggagggaagggggggtcatattctcaggctgcatgttcagattctgctcaaggtagtgacgtaagtttgacagcctga